(19) United States
(12) Patent Application Publication
(10) Pub. No.: US 2022/0372159 A1
(43) Pub. Date: Nov. 24, 2022

(54) Treatment and Prevention of Ischemic Diseases And/Or Ischemic Tissue Damages

(71) Applicant: Julius-Maximilians-Universitaet-Wuerzburg, Wuerzburg (DE)

(72) Inventors: Michael Schuhmann, Wuerzburg (DE); Guido Stoll, Rimpar (DE); Bernhard Nieswandt, Eibelstadt (DE); David Stegner, Wuerzburg (DE)

(73) Assignee: Julius-Maximilians-Universitaet-Wuerzburg, Wuerzburg (DE)

(21) Appl. No.: 17/624,043

(22) PCT Filed: Jul. 1, 2020
(86) PCT No.: PCT/EP2020/068464

§ 371 Date: Dec. 30, 2021

(30) Foreign Application Priority Data
Jul. 1, 2019 (EP) 19183678.2

(51) Int. Cl. C07K 16/28; A61P 9/10; A61K 39/395

(52) U.S. Cl. CPC C07K 16/2896; A61P 9/10; A61K 39/3955; A61K 2039/505

(57) ABSTRACT

The invention relates to a CD84 inhibitor for use in treating or preventing of an ischemic disease and/or ischemic tissue damage, the use of a CD84 inhibitor for inhibiting CD84 mediated T cell migration in vitro and a method for screening for a CD84 inhibitor.

The invention relates inter alia to a CD84 inhibitor for use in treating or preventing of an ischemic disease and/or of an ischemic tissue damage, the use of a CD84 inhibitor for inhibiting CD84 mediated T cell migration in vitro, and a method for screening for a CD84 inhibitor.

### BACKGROUND OF THE INVENTION

Ischemia is an interruption in blood supply to tissues, causing a shortage of oxygen with resultant damage to tissue. It usually also involves local anemia in a given part of a body and is associated with reduced availability of nutrients and inadequate removal of metabolic wastes. Ischemic tissue damages can lead, if untreated, to tissue death. Ischemic tissue damages can also give rise to ischemic diseases.

Ischemic diseases and ischemic tissue damages are usually caused by problems with blood vessels, with resultant damage to or dysfunction of tissue. They may be caused e.g. by embolism, transplantation, thrombosis of an atherosclerotic artery, or trauma. Venous problems like venous outflow obstruction and low-flow states can cause acute arterial ischemia. An aneurysm is one of the most frequent causes of acute arterial ischemia. Other causes are e.g. heart conditions including myocardial infarction, mitral valve disease, chronic atrial fibrillation, cardiomyopathies, and prosthesis, in all of which thrombi are prone to develop.

For example, brain ischemia, also known as stroke, is one of the major causes of death and invalidity worldwide with limited treatment options. In 2015, stroke was the second most frequent cause of death after coronary artery disease, accounting for 6.3 million deaths (11% of the total). About 3.0 million deaths resulted from ischemic stroke while 3.3 million deaths resulted from hemorrhagic stroke. About half of people who have had a stroke live less than one year.

In acute ischemic stroke the primary therapeutic goal is reconstitution of cerebral blood flow, achievable either by treatment with recombinant tissue-type plasminogen activator (rt-PA) and/or mechanical thrombectomy (MTE) in particular after occlusion of major blood-supplying vessels such as the intracranial internal (ICA) and/or middle cerebral artery (MCA) (Berkhemer et al., Global Burden Disease study). Despite recent advances by MTE with high recanalization rates of up to 80%, infarction development often progresses during reperfusion (Mizuma et al.) resulting in a considerably high number needed to treat for a good outcome (1:4-6) and many cases with futile recanalization (Goyal et al., Church et al.).

Ongoing infarct development is attributable to ischemia/reperfusion (I/R) injury, which has since the 1980ies been recognized as the harmful component of blood flow return after transient organ ischemia such as the brain, heart, kidney and liver (Mizuma et al., Hallenbeck et al., Eltzschig et al.). Further, reperfusion injury may also occur after organ transplantation, when blood flow is restored in the recipient. T cells contribute to I/R injury in the brain in an antigen-independent manner as immuno-deficient Rag1^{-/-} mice were protected against I/R injury in the transient middle cerebral artery occlusion (tMCAO) model, while adoptive transfer (AT) of T cells fully reconstituted stroke susceptibility (Yilmaz et al., Kleinschnitz et al. 2010). Besides T cells, platelets have been identified as key orchestrators of cerebral I/R injury, that promote inflammation and thrombotic activity in the microcirculation that can efficiently be prevented by blocking early steps of platelet adhesion and activation via glycoprotein (GP)lb and GPVI receptors, respectively (Kleinschnitz et al. 2007). The pathogenic effect of platelets is linked to that of T cells as depletion of platelets in Rag1^{-/-} mice abolished the detrimental effect of adoptively transferred T cells (Kleinschnitz et al. 2013). These findings led to the recent concept of thrombo-inflammation as the driving force underlying I/R injury in the ischemic brain (Stoll et al. 2010, Stoll et al. 2019, Nieswandt et al.). Thereby platelets are believed to act in concert with T cells to cause further brain injury but the underlying mechanisms and the molecular link between the two cell types have remained elusive.

Cluster of differentiation 84 (CD84) is a member of the signaling lymphocyte activation molecule (SLAM) family and acts as a homophilic cell adhesion molecule highly expressed on different immune cell populations and platelets (Nanda and Phillips 2006, Martin et al., Romero et al.). The N-terminal ectodomain of CD84 mediates the homophilic interaction between CD84 proteins (Martin et al., Romero et al.), while the C-terminal intracellular portion of CD84 bears two immunoreceptor tyrosine-based switch motifs (ITSM), which can bind the intracellular adapters SLAM-associated protein (SAP also termed SH2D1A) and Ewing's sarcoma activated transcript 2 (EAT-2) (Nanda et al. 2005, Tangye et al.). CD84-ligation triggers ITSM phosphorylation and SAP recruitment, resulting in enhanced interferon (IFN) γ production and proliferation in T cells stimulated with low doses of anti-CD3 antibodies (Nanda and Phillips 2006, Tangye et al.). CD84 has been established as functional co-receptor in lymphocytes that facilitates prolonged B cell:T cell interactions required for germinal center formation (Cannons et al.).

Thus, while CD84 appears to have a role in immune cell activation, its function in platelets is not well understood (see also Hofmann 2013). Upon platelet activation CD84 is shed from the surface (Hofmann et al. 2012), but the (patho-) physiological significance of soluble CD84 is unknown. Notably, CD84-deficiency had no effect on the hemostatic or thrombotic platelet activity in mice in vitro and in vivo (Hofmann et al. 2014).

As mentioned above, so far several methods for treating ischemic diseases were developed. However, the number of deaths due to an ischemic disease remains still high as well as the number of patients suffering from complications, e.g. due to ischemia/reperfusion (I/R) injury. Moreover, the molecular mechanisms underlying ischemic tissue damage and corresponding ischemic diseases are still elusive.

The object of the present invention is to provide a treatment for ischemic diseases and/or ischemic tissue damages.

### SUMMARY OF THE INVENTION

A CD84 inhibitor for use in treating or preventing of an ischemic disease and/or of an ischemic tissue damage is provided.

The invention further relates to a use of a CD84 inhibitor for inhibiting CD84 induced T cell migration in vitro.

Moreover, a method for screening for a CD84 inhibitor, the method comprising identifying whether a potential CD84 inhibitor inhibits T cell migration is provided.

### DETAILED DESCRIPTION OF THE INVENTION

In a first aspect, the invention relates to a CD84 inhibitor for use in treating or preventing of an ischemic disease and/or for use in treating or preventing of an ischemic tissue damage, in particular for use in treating or preventing of ischemic stroke.

As shown in the examples, CD84 is an important factor for T cell migration for ischemic tissue damages. This also makes this factor attractive as a target for the treatment of ischemic diseases.

As used herein, the term “inhibitor” is a molecule that induces the partial or complete suppression, inhibition or blocking of the expression, activity or interaction of CD84. Inhibition of activity may also be an inhibition or reduction of CD84-mediated migration of T cells. Not every molecule that binds to CD84 is an inhibitor. An antibody that binds to CD84 (anti-CD84 antibody) may be an inhibitor or a non-inhibitor. It is only an inhibitor if it induces the partial or complete suppression, inhibition or blocking of the expression, activity or interaction of CD84 (blocking antibody). The CD84 inhibitor as used herein is further defined below.

As used herein, the terms “suppression”, “inhibition” or “blocking” are used interchangeably.

Preferably, the inhibitor partially or completely inhibits the interaction of a CD84 molecule with another CD84 molecule.

As used herein, the term “CD84 molecule” is a molecule that comprises Cluster of Differentiation 84 (CD84 protein), preferably as a cell surface molecule, or any chemical version of this CD84 protein, e.g. a CD84 gene or a CD84 RNA. Examples of “CD84 molecules” are given by sequences of SEQ ID NO: 1 and SEQ ID NO: 2. There may be different types of CD84 molecules, e.g. platelet derived soluble CD84 versus CD84 surface molecules of T cells.

Preferably, the CD84 inhibitor inhibits the expression, activity or interaction of CD84 by at least 10%, at least 20%, at least 30%, at least 35%, at least 40%, at least 45%, at least 50%, at least 55%, at least 60%, at least 65%, at least 70%, at least 75%, at least 80%, at least 85%, at least 90% or by at least 95%.

Methods for to detecting the efficiency of the binding of CD84 binding molecules to CD84 are well known to the person skilled in the art and include immunoassays, such as enzyme-linked immunosorbent assay (ELISA), Western Blot, enzyme-linked immuno spot assay (ELISPOT) and Indirect Immunoperoxidase Assay.

The CD84 inhibitor preferably inhibits a CD84 molecule by binding to CD84. The inhibitor may bind to any position of the extracellular part of a CD84 sequence, for example, it may bind to the IgV domain or IgV2 domain of CD84. Preferably the inhibitor binds to a subsection of CD84 as shown in SEQ ID NO: 3. In some embodiments the inhibitor binds to CD84 as shown in SEQ ID NO: 1.

Preferably, the CD84 inhibitor may inhibit the interaction between one CD84 molecule and another CD84 molecule, e.g. two different types of CD84 molecules. Said inhibition may be by direct interaction with at least one of the dimer interfaces of one of these CD84 molecules, i.e. interfaces that effect the homotypic-receptor interaction of two CD84 molecules and/or contact during dimerization. In some embodiments the CD84 inhibitor inhibits the interaction of CD84 on T cells with another CD84 molecule derived from platelets, in particular with soluble CD84 (sCD84) derived from platelets, preferably homotypic-receptor interaction with soluble CD84 (sCD84) derived from platelets. In some embodiments a “dimer” or “dimerization” may also refer to a receptor-receptor interaction, in particular between a CD84 of a T cell and soluble CD84.

In another preferred embodiment, the CD84 inhibitor is capable of inhibiting dimerization of CD84 molecules, in particular dimerization of a CD84 of a T cell with soluble CD84.

The CD84 inhibitor may inhibit the interaction of CD84 by interacting with at least one of the dimer interfaces of CD84. These include positions 51, 53, 108, 110 or 112 of SEQ ID NO: 1 or positions 51, 53, 106, 108 or 110 of SEQ ID NO: 2. Preferably, the CD84 inhibitor interacts with at least one of the positions 51, 53, 108, 110 or 112 of SEQ ID NO: 1 or positions 51, 53, 106, 108 or 110 of SEQ ID NO: 2. Preferably, the CD84 inhibitor interacts with at least one of the positions 21, 23, 78, 80 or 82 of SEQ ID NO: 3 or positions 21, 23, 76, 78 or 80 of SEQ ID NO: 4.

In some embodiments the CD84 inhibitor interacts with SEQ ID NO: 3 of human CD84 or SEQ ID NO: 4 of mouse CD84. Preferably, the CD84 inhibitor may inhibit the interaction between one CD84 molecule to another CD84 molecule by direct interaction with at least one of the positions 21, 23, 78, 80 or 82 of SEQ ID NO: 3.

Preferably, the CD84 inhibitor may inhibit the interaction between one CD84 molecule to another CD84 molecule by direct interaction with at least one of the positions 51, 53, 108, 110 or 112 of SEQ ID NO: 1.

Further, the CD84 inhibitor may inhibit the interaction between one CD84 molecule and another CD84 molecule sterically by interacting with a site of a CD84 molecule, which is not a dimer interface.

The type of interaction of the CD84 inhibitor with a CD84 protein molecule may be any kind of interaction known to a person skilled in the art, such as a covalent bond, an ionic bond, a hydrogen bridge bond, a dipol-dipol interaction, a hydrophobic interaction and/or a van der Waals interaction. Preferably, the type of interaction of the CD84 inhibitor with a CD84 protein molecule is a non-covalent bond, in particular an ionic bond, a hydrogen bridge bond, a hydrophobic interaction and/or a van der Waals interaction. For example, a hydrogen bridge bond may be formed between an oxygen atom and a nitrogen atom, e.g. in case the CD84 inhibitor is an antibody. In some embodiments, the CD84 inhibitor is capable of reversibly binding to a CD84 protein molecule.

As used herein, the inhibition refers to the inhibition of any variant of CD84, e.g. a CD84 protein, a CD84 RNA, a CD84 gene. CD84 may be derived from animals or humans.

These may further be modified in any way. This modification may comprise any RNA modification known to the person skilled in the art, such as splicing variants, as well as any protein modification known to a person skilled in the art, such as any known chemical modification, e.g. posttranslational modifications. Further, this may also comprise any shortened version, which was e.g. cut by a protease, such as ADAM10.

Further, the inhibitor may interfere with any process resulting in the formation of protein CD84. This may comprise the inhibition of the transcription of a CD84 gene or the inhibition of the formation of CD84 RNA as well as the inhibition of the translation of CD84 RNA or the inhibition of the formation of CD84 protein.

An example for a CD84 that may be subject of inhibition by a CD84 inhibitor according to the first aspect may be a protein having the sequence of SEQ ID NO: 1 or SEQ ID NO:2.

Preferably, the CD84 that may be subject of inhibition by a CD84 inhibitor according to the first aspect has a sequence, which is at least 50%, at least 55%, at least 60%, at least 65%, at least 70%, at least 75%, at least 80%, at least 85%, at least 90%, at least 95%, or 100% (e.g., at least 86%, at least 87%, at least 88%, at least 89%, at least 91%, at least 92%, at least 93%, at least 94%, at least 95%, at least 96%, at least 97%, at least 98% or at least 99%) identical to SEQ ID NO: 1 or SEQ ID No: 2. Preferably, the CD84 protein has the sequence of SEQ ID NO: 1 or SEQ ID NO: 2.

Preferably the CD84 inhibitor binds to SEQ ID NO: 3 of human CD84 or to SEQ ID NO: 4 of mouse CD84. Preferably, the CD84 that may be subject of inhibition by a CD84 inhibitor according to the first aspect comprises a sequence section, which is at least 50%, at least 55%, at least 60%, at least 65%, at least 70%, at least 75%, at least 80%, at least 85%, at least 90%, at least 95%, or 100% (e.g., at least 86%, at least 87%, at least 88%, at least 89%, at least 91%, at least 92%, at least 93%, at least 94%, at least 95%, at least 96%, at least 97%, at least 98% or at least 99%) identical to SEQ ID NO: 3 or SEQ ID No: 4. Preferably, the CD84 protein comprises the sequence section of SEQ ID NO: 3 or SEQ ID NO: 4.

Further, preferably, the CD84 that may be subject of inhibition by a CD84 inhibitor according to the first aspect has a sequence, which is at least 50%, at least 55%, at least 60%, at least 65%, at least 70%, at least 75%, at least 80%, at least 85%, at least 90%, at least 95%, or 100% (e.g., at least 86%, at least 87%, at least 88%, at least 89%, at least 91%, at least 92%, at least 93%, at least 94%, at least 95%, at least 96%, at least 97%, at least 98%, at least 99% or preferably 100%) identical to a shortened version of SEQ ID NO: 1 or SEQ ID No: 2 comprising the amino acids from position 22 to 197 and 22 to 221, respectively.

Further, preferably, the CD84 that may be subject of inhibition by a CD84 inhibitor according to the first aspect has a sequence, which is at least 50%, at least 55%, at least 60%, at least 65%, at least 70%, at least 75%, at least 80%, at least 85%, at least 90%, at least 95%, or 100% (e.g., at least 86%, at least 87%, at least 88%, at least 89%, at least 91%, at least 92%, at least 93%, at least 94%, at least 95%, at least 96%, at least 97%, at least 98%, at least 99% or preferably 100%) identical to a shortened version of SEQ ID NO: 1 or SEQ ID No: 2 comprising the amino acids from position 31 to 129 and 31 to 126, respectively.

The term “at least 50% amino acid identity” as used herein means that the amino acid sequence of the CD84 that may be subject of inhibition by a CD84 inhibitor according to the first aspect has an amino acid sequence characterized in that, within a stretch of 100 amino acids, at least 50 amino acid residues are identical to the sequence of the corresponding sequence, e.g. of SEQ ID No: 1, SEQ ID No: 2, SEQ ID No: 3 or SEQ ID No: 4.

Sequence identity can, e.g., be determined by methods of sequence alignment in form of sequence comparison. Methods of sequence alignment are well known in the art and include various programs and alignment algorithms. Moreover, the NCBI Basic Local Alignment Search Tool (BLAST) is available from several sources, including the National Center for Biotechnology Information (NCBI, Bethesda, Md.) and on the internet, for use in connection with the sequence analysis programs blastp, blastn, blastx, tblastn and tblastx. Percentage of identity of mutants relative to the amino acid sequence of e.g. SEQ ID No: 1, SEQ ID No: 2, SEQ ID No: 3 or SEQ ID No: 4 is typically characterized using the NCBI Blast blastp with standard settings. The comparison of sequences and determination of percent identity between two amino acid sequences can also be accomplished with the program “BLAST 2 SEQUENCES (blastp)” (Tatusova et al.) with the following parameters: Matrix BLOSUM62; Open gap 11 and extension gap 1 penalties; gap x_dropoff50; expect 10.0 word size 3; Filter: none.

Alternatively, sequence identity may be determined using the software GENEious with standard settings. Alignment results can be, e.g., derived from the Software Geneious (version R8), using the global alignment protocol with free end gaps as alignment type, and Blosum62 as a cost matrix.

According to some embodiments, the sequence comparison may cover at least 100 amino acids, more preferably at least 120 amino acids.

Various CD84 inhibitors may be used in the context of the present invention.

In a preferred embodiment, the CD84 inhibitor for use of the first aspect as described herein may be selected from the group consisting of an antibody, a blocking polypeptide, a small molecule, or an inhibitory nucleic acid.

In particularly preferred embodiments, the CD84 inhibitor is an antibody. Typically and as generally known in the art, an antibody is a protein belonging to the protein family of immunoglobulins and is composed in its variable regions of framework regions (subdivision of the variable region (Fab) of an antibody) and complementarity determining regions (CDRs). Naturally, antibodies are produced by plasma cells in response to a certain antigen. In general, each antibody has two identical heavy chain immunoglobulins and two identical light chain immunoglobulins. Each heavy and each light chain may have a variable and a constant region. The constant region of a heavy chain may be one of five types of mammalian Ig heavy chains: α, δ, ε, γ and μ. The type of the heavy chain present usually defines the class (isotype) of the antibody: IgA, IgD, IgE, IgG and IgM antibodies, respectively. Similarly, the constant region of a light chain may be one of two types of mammalian Ig light chains: κ and λn. The variable regions of heavy and light chains are usually made of a unique combination of numerous protein sequences allowing the binding to a particular antigen.

The term “antibody” also covers an isolated antibody. In some embodiments, the antibody is an isolated antibody.

In general, each heavy chain is connected to one of the light chains, whereby the variable regions of a heavy and a light chain combine to form one of the two identical antigen-binding sites and their constant regions combine to form the constant region of the antibody. Further, both constructs of one heavy and one light chain may be connected via the constant regions of their heavy chains, forming a “Y”-shaped molecule, whereby the two arms depict the antigen-binding variable region and the stem depicts the constant region.

The antibody may be a complete antibody, meaning that it usually comprises a heavy chain of three or four constant domains and a light chain of one constant domain as well as the respective variable domains, whereby each domain may comprise further modifications, such as mutations, deletions or insertions, which do not change the overall domain structure.

Further, the antibody may form a homo- or heterodimer or a homo- or heteromultimer, whereby “dimer” and “multimer” means that two and at least three antibodies, respectively, may combine to form a complex. The prefix “homo” means that a complex may be formed of identical antibody molecules, whereby the prefix “hetero” means that a complex may be formed of different antibody molecules.

Similarly, “dimers” of CD84 may refer to homo- or heterodimers, preferably heterodimers of different types of CD84 molecules, e.g. platelet derived CD84 and CD84 bound to T cells.

In general, the term “antibody” is intended to comprise all above-mentioned immunoglobulin isotypes, i.e. the antibody may be an IgA, IgD, IgE, IgG or IgM antibody, including any subclass of these isotypes. Preferably, the antibody is an IgG antibody. Since the antibody may be expressed and produced recombinantly, the antibody may also comprise two different constant regions of heavy chains, e.g. one IgG1 and one IgG2 heavy chain, or heavy chains from different species. However, the heavy chains preferably are from the same species. Furthermore, the antibody may comprise either a lambda or a kappa light chain.

Usually, the antibody may refer to a monoclonal, a bispecific or a multispecific antibody. Such antibodies are known in the art. As used herein, the term “monoclonal” may be understood in the broadest sense describing antibodies produced by a single clone of B lymphocytes or antibodies having the same or a similar amino acid sequence. The term “bispecific”, as used herein, may be understood in the broadest sense describing antibodies interacting with two different epitopes. The bispecific antibody may be derived from two monoclonal antibodies. Optionally, these two different epitopes may be localized on the same antigen, but they may also be localized on two different antigens. The term “multispecific”, as used herein, may be understood in the broadest sense describing antibodies interacting with three or more different types of epitopes. Optionally, these epitopes may be localized on the same antigen or on two or more antigens.

Preferably, the antibody according to aspect two is a monoclonal antibody. Further, the antibody according to aspect two preferably is a bispecific or a multispecific antibody.

Methods for the production of antibodies in general are well known to the person skilled in the art. Preferably, antibodies are produced by making hybridoma cells. Methods for the production of hybridoma cells as well as methods for the production of antibodies with the help of hybridoma cells are well known to the person skilled in the art. Generally, mice are injected with the desired antigen and killed after a few days in order to isolate the spleen cells secreting the antibody against the desired antigen. In general, fusion of these antibody-secreting spleen cells with immortal non-secreting myeloma cells results in hybridoma cells. These hybridoma cells are then usually screened and the hybridoma producing the desired antibody is selected. The selected hybridoma may then be cultured in vivo or in vitro and antibodies can be isolated. However, this method does not necessarily lead to antibodies that are CD84 inhibitors, i.e. which inhibit CD84 activity. A relevant part for the production of antibodies is to select such antibodies that actually inhibit CD84 activity. Often antibodies do not inhibit CD84 activity, in particular homotypic interactions of CD84 molecules. Surprisingly, the discovery of the inventors leads to a new selection assay that allows for separation of antibodies that do not inhibit CD84 activity from antibodies that actually inhibit CD84 activity. The assay is based on the effect of antibodies upon the migration speed of stimulated T cells. Further details are given below and are provided in example 8.

Bifunctional, or bispecific, antibodies may have antigen-binding sites of different specificities. Various forms of bispecific antibodies and their production are known to the person skilled in the art. For example, these include BSIgG, which are IgG molecules comprising two distinct heavy chains and two distinct light chains that are secreted by so-called “hybrid hybridomas”, and heteroantibody conjugates produced by the chemical conjugation of antibodies or antibody fragments of different specificities (Segal et al., Van Spriel et al.).

Bispecific antibodies may be generated to deliver cells, cytotoxins, or drugs to specific sites. An important use may be to deliver host cytotoxic cells, such as NK or cytotoxic T cells, to specific cellular targets. (Lachmann. et al.). Another important use may be the delivery of cytotoxic proteins to specific cellular targets (Raso and Griffin; Honda et al.). A further important use may be to deliver anti-cancer non-protein drugs to specific cellular targets (Corvalan et al. Pimm et al.). Such bispecific antibodies may be prepared by chemical cross-linking (Brennan et al.), disulfide exchange, or the production of hybrid-hybridomas (quadromas). Quadromas may be constructed by fusing hybridomas that secrete two different types of antibodies against two different antigens (Milstein and Cuello).

As used herein, the term “blocking polypeptide” refers to a peptide comprising an amino acid sequence, which can be recognized and bound by a CD84 protein and induces the partial or complete suppression, inhibition or blocking of the expression, activity or interaction of CD84. The blocking peptide may have a sequence of 5 to 100 covalently bound amino acids. Preferably, the blocking polypeptide comprises 10 to 90, more preferably 20 to 80, even more preferably 30 to 70 and especially preferred 40 to 60 amino acids. The blocking polypeptide may comprise proteinogenic, non-proteinogenic and/or non-natural amino acids. Moreover, the blocking polypeptide may comprise post-translational modifications, such as phosphorylation or glycosylation.

A blocking polypeptide can be isolated and purified from a natural source. Methods for isolating and purifying a blocking polypeptide from a natural source are well known to the person skilled in the art. For example, the blocking polypeptide can be purified using a chromatography technique, such as high performance liquid chromatography (HPLC) or ion exchange chromatography, gel filtration, or other known methods. Alternatively, a blocking poylpeptide can be chemically synthesized by conventional methods. Methods for synthesizing blocking polypeptides are well known to the person skilled in the art. Furthermore, the blocking polypeptide may be produced by recombinant peptide expression, e.g. in bacterial, yeast or insect cells, and isolated as well as purified as described above.

As used herein, the term “small molecule” refers to molecules having a molecular mass of up to 800 g/mol, preferably 600 g/mol, more preferably 400 g/mol and most preferably 200 g/mol. A small molecule may be a chemical substance of any biochemical substance class, such as peptides or nucleic acids. Further, a small molecule may also belong to a substance class of organic chemistry.

As used herein, the term “inhibitory nucleic acid” refers to a nucleic acid comprising a nucleic acid sequence that binds to a nucleic acid sequence, which encodes a CD84 protein. The inhibitory nucleic acid may have a sequence of 10 to 100 covalently bound nucleic acids. Preferably, the inhibitory nucleic acid comprises 20 to 90, more preferably 30 to 80, even more preferably 40 to 70 and especially preferred 50 to 60 nucleic acids. The inhibitory nucleic acid may comprise DNA and/or RNA molecules. Moreover, the inhibitory nucleic acid may comprise modifications, such as methylations.

An inhibitory nucleic acid can be isolated and purified from a natural source. Methods for isolating and purifying an inhibitory nucleic acid from a natural source are well known to the person skilled in the art. For example, the inhibitory nucleic acid can be purified using a chromatography technique, such as high performance liquid chromatography (HPLC) or ion exchange chromatography, gel filtration, or other known methods. Alternatively, an inhibitory nucleic acid can be chemically synthesized by conventional methods. Methods for synthesizing inhibitory nucleic acids are well known to the person skilled in the art.

Preferably, the inhibitory nucleic acid sequence is an aptamer. Aptamers are oligonucleotide or peptide molecules that bind to a specific target molecule. Aptamers are usually created by selecting them from a large random sequence pool.

In a more preferred embodiment, the CD84 inhibitor antibody for use of the first aspect may be selected from the group consisting of a monoclonal antibody, a chimeric antibody, a humanized antibody, a human antibody, a bispecific antibody, an scFv, a multimer of an scFv, a tandab, a diabody, triabody, a flexibody, or a fragment thereof.

In general, a chimeric antibody is an antibody made by combining genetic material from two different species, for example from a nonhuman source, like a mouse, with genetic material from a human being.

In general, humanized antibodies are a particular type of chimeric antibodies. For example, humanized antibodies may be produced by grafting DNA of a human antibody into the mouse antibody framework coding DNA or by grafting DNA of a mouse antibody into human antibody framework coding DNA. Preferably, DNA of a human antibody is grafted into the mouse antibody framework coding DNA. In general, grafting of DNA comprises grafting of one or more DNA sequences into the target antibody framework coding DNA. Optionally, the variable and constant regions as well as heavy and light chains may be partially or fully humanized. Preferably, the heavy chain variable region and the light chain variable region of a mouse antibody are humanized. More preferably, the heavy chain variable region and the light chain variable region of a mouse antibody are humanized by changing a DNA sequence encoding 1 to 50, preferably, 1 to 30, more preferably 1 to 20 amino acids. The DNA grafted may generally comprise DNA regions of the six hypervariable loops determining antigen specificity, also called complementarity-determining regions (CDR), or DNA regions not comprising a CDR, or both. Preferably, the humanization comprises grafting of DNA not comprising CDRs.

In general, the resulting DNA construct may then be used to express and produce antibodies that are usually less or not immunogenic in comparison to the non-human parental antibody. This includes the production of modified antibodies such as glycosylated antibodies or fucosylated antibodies. Such methods are well known in the art.

In general, a human antibody is an antibody made by genetic material of a human being only.

In a scFv, the two antigen-binding variable regions of the light and heavy chain (VH Fv and VL Fv) of an antibody are in general artificially connected by a linker peptide, designated as single chain variable fragment or single chain antibody (Bird et al., Orlandi et al., Clarkson et al.). The antigen-binding site can be made up of the variable domains of light and heavy chains of a monoclonal antibody. Several investigations have shown that the scFv fragment may have indeed the full intrinsic antigen-binding affinity of one binding site of the whole antibody.

As used herein, diabodies are scFv with two binding specificities and can either be monospecific and bivalent or bispecific and bivalent.

As used herein, tribodies are scFv with three binding specificities and can either be monospecific and bivalent, bispecific and bivalent or trispecific and bivalent.

Tandabs and flexibodies are further antibody formats, which are e.g. defined in US2007031436 and EP1293514B1, respectively.

Antibody fragments that contain the idiotypes of the protein can be generated by techniques known in the art. For example, such fragments include, but are not limited to, the F(ab′)2 fragment which can be produced by pepsin digestion of the antibody molecule; the Fab′ fragment that can be generated by reducing the disulfide bridges of the F(ab′)2 fragment; the Fab fragment that can be generated by treating the antibody molecular with papain and a reducing agent; and Fv fragments.

As claimed herein, the CD84 inhibitor is used to treat or prevent an ischemic tissue damage, and/or ischemic disease.

The term “treating” as used herein means the slowing, interrupting, arresting or stopping of the progression of an ischemic tissue damage, and/or ischemic disease or a condition thereof, and does not necessarily require the complete elimination of all disease symptoms and signs. Treatment is possible after onset of the ischemic tissue damage.

The term “preventing” as used herein means any degree of inhibition of the time of onset or severity of signs or symptoms of an ischemic tissue damage and/or an ischemic disease or a condition thereof, including, but not limited to, the complete prevention of the disease or condition.

For example, administration of a CD84 inhibitor according to the first aspect may result in reduced infarct volumes in comparison to an untreated organ or untreated subject. Preferably, infarct volumes are reduced by at least 5%, at least 8%, at least 10%, at least 12%, at least 15%, at least 20%, at least 25%, at least 30%, at least 40%, at least 50% or by at least 60%.

The administration of the CD84 inhibitor may result in a better clinical outcome as revealed by an improved National Institutes of Health Stroke Scale (NIHSS). The NIHSS usually quantifies stroke severity based on the weighted evaluation of several different health parameters investigated in a subject, such as testing of the field of vision. The sum of the values from the investigations results in a maximum of 42 points (the higher the score, the more extensive the stroke). Recanalization therapy for ischemic infarction is generally indicated for a NIHSS score above 6 points and below 22 points. The CD84 inhibitor may be applied at an NIHSS from 6 to 25, preferably from 8 to 23, more preferably from 10 to 20, even more preferably from 12 to 17 and most preferred from 14 to 16. In general, the administration of the CD84 inhibitor may result in a reduction oft he NIHHS of at least 1 point, at least 2 points, at least 3 points, at least 4 points, at least 5 points, at least 6 points, at least 7 points, at least 8 points, at least 9 points, at least 10 points, at least 11 points, at least 12 points, at least 13 points, at least 14 points, at least 15 points, at least 16 points, at least 17 points, at least 18 points, at least 19 points, at least 20 points, at least 22 pints or at least 25 points.

For example, administration of a CD84 inhibitor according to the first aspect may further result in reduced numbers of CD4+ T cells in the infarcted tissue, such as brain tissue, in comparison to an untreated organ or untreated subject. Preferably, the numbers of CD4+ T cells in the infarcted area are reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or by at least 95%.

In another example, administration of a CD84 inhibitor according to the first aspect may also result in reduced percentages of occluded vessels in the ipsilateral hemisphere in comparison to an untreated cell or untreated subject. Preferably, the percentages of occluded vessels in the ipsilateral hemisphere are reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or by at least 95%.

Further, CD84 generally may also be involved in the process of forming or continuation of occlusion occurring during an ischemic disease. Therefore, the CD84 inhibitor according to the first aspect may also be used in treating or preventing of an occlusion during an ischemic disease.

To achieve these effects, the CD84 inhibitor may be administered in a therapeutically effective amount. A therapeutically effective amount of the substances results in a beneficial effect for the treated individual. Such an amount can be determined based on age, race, sex, and other factors based on the individual subject to be treated.

As used herein, the term “ischemic tissue damage” refers to tissue damage caused by a lack of oxygen. An ischemic disease is usually associated with ischemic tissue damage. In some embodiments, the ischemic tissue damage may also be an ischemia/reperfusion (I/R) injury due to an organ transplantation. Organs affected by an ischemic tissue damage are well known to the person skilled in the art. For example, the ischemic tissue damage may occur in the heart, brain, lung, spleen, limb, bone, testicle, eye or bowel.

As used herein, the term “ischemic disease” refers to a disease caused and/or characterized by reduced blood (and hence oxygen) supply to the diseased tissue/organ. The reduced blood flow may be totally or partly reduced, resulting in a partial (poor perfusion) or total ischemic disease. The ischemic disease may further be an acute ischemic disease or a chronic ischemic disease.

Types of ischemic diseases and/or ischemic tissue damages are well known to a person skilled in the art. Non-limiting examples of ischemic diseases include trauma, ischemic cerebrovascular disorder (such as apoplexy or cerebral infarction), ischemic renal disease, ischemic pulmonary disease, infection-related ischemic disease, ischemic disease of limbs, ischemic heart disease, myocardial infarction, atherosclerosis, peripheral vascular disorder, a pulmonary embolus, a venous thrombosis, a transient ischemic attack, unstable angina, cerebral vascular ischemia, stroke, an ischemic neurological disorder, ischemic kidney disease, vasculitis, transplantation, e.g. kidney or liver transplantation, in particular I/R injury due to transplantation, endarterectomy, aneurysm repair surgery, an inflammatory disorder, hypothermia or traumatic injury. Ischemic tissue damage may be due to an ischemic disease or, alternatively, due to an external injury that is not directly caused by a disease, e.g. a reperfusion injury after kidney or liver transplantation. In a preferred embodiment, the disease is cerebral ischemia and/or cerebral infarction and/or a thrombo-inflammatory disease, preferably ischemic stroke.

The subject to be treated with the CD84 inhibitor according to the first aspect may be a human or animal, such as a mammal. The subject may be an otherwise healthy individual or may exhibit further diseases and/or co-morbidities, and/or is treated with medication(s) for further diseases and/or co-morbidities. The subject, in addition to an ischemic tissue damage and/or an ischemic disease, may exhibit further diseases, and/or co-morbidities, and/or is treated with medication(s) for further diseases and/or co-morbidities.

In a further preferred embodiment, the CD84 inhibitor for use of the first aspect may inhibit the interaction of CD84 on T cells with another CD84 molecule derived from platelets. The CD84 inhibitor may bind to CD84 on T cells and/or CD84 molecule derived from platelets, in particular to both.

In a further preferred embodiment, the method further comprises suppression of T cell migration with said CD84 inhibitor, in particular wherein the ischemic disease and/or ischemic tissue damage is at least partially mediated by T cell migration

As used herein, the term “T cell” refers to a type of lymphocyte presenting a T-cell receptor on the cell surface. Usually, T cells develop in the thymus gland and play an important role in the immune response. In general, T cells originate as precursor cells, derived from bone marrow, and develop into several distinct types of T cells once they have migrated in to the thymus gland. A T cell may be a conventional adaptive T cell or an innate-like T cell. Examples for conventional adaptive T cells are helper CD4⁺ T cells, cytotoxic CD8⁺ T cells, memory T cells and regulatory CD4⁺ T cells. Preferably, the T cell is a CD4⁺ cell, more preferably, the T cell is a helper CD4⁺ T cell and/or a regulatory CD4⁺ T cell. Examples of innate-like T cells are natural killer T cells, mucosal associated invariant or gamma delta T cells.

In general, the CD84 on T cells may be a CD84 protein anchored in the cellular membrane of a T cell via its transmembrane domain.

As used herein, the term “platelet” refers to a discoid-shaped anucleate cell fragment that is usually produced by megakaryocytes (MKs) in the bone marrow. In general, due to the lack of a nucleus, platelets are only to a limited extent capable of de novo protein synthesis, using MK-derived mRNA and translational machinery. In general, the pivotal primary hemostatic function of platelets is only retrieved upon injury, when platelets in the flowing blood come into contact with the exposed subendothelial extracellular matrix (ECM), which leads to their rapid activation and the formation of a hemostatic plug. This lifesaving function of platelets limits blood loss following injury. On the other hand, under pathological conditions, like rupture of an atherosclerotic plaque, platelet aggregation may lead to formation of occlusive thrombi resulting in vessel occlusion and infarction of vital organs. Therefore, platelet activation has to be tightly regulated, which requires a complex interplay of adhesion and activation receptors, release of soluble mediators, inhibitory receptors, as well as cleavage and inactivation of receptors, in order to facilitate stable aggregate formation to seal lesions, while preventing excessive thrombus formation.

In general, the CD84 derived from a platelet means a soluble portion of a CD84 protein molecule (sCD84), which was anchored in the cellular membrane of a platelet via its transmembrane domain and then shed e.g. by proteolytic cleavage. Preferably, the soluble portion of a CD84 protein molecule was received after shedding by a protease, more preferably by a metalloproteinase, even more preferably by the metalloproteinase Adam10. The soluble portion of a CD84 protein molecule may comprise the amino acids of positions 31 to 197, preferably the amino acids of positions 31 to 180, more preferably the amino acids of positions 31 to 150, even more preferably the amino acids of positions 31 to 120 and most preferably the amino acids of positions 31 to 90 of SEQ ID NO: 1 or SEQ ID NO: 2.

In another preferred embodiment, the CD84 inhibitor for use of the first aspect may bind to the domain of CD84 that mediates homotypic receptor interaction of CD84.

The term “homotypic receptor interaction of CD84” as used herein refers to interactions of CD84 with another similar structure, in particular interactions via their dimer interface. Preferably, this other similar structure is a second CD84 protein molecule, more preferably homotypic receptor interaction refers to interaction of a platelet derived and/or soluble CD84 with a CD84 protein anchored in a cellular membrane, in particular of a T cell. Dimerization may refer to such a homotypic receptor interaction of CD84.

In a further preferred embodiment, the CD84 inhibitor for use of the first aspect may be used in treating or preventing ischemia/reperfusion injury and/or infarct growth.

The term “ischemic/reperfusion injury” as used herein refers to tissue damage caused when blood supply returns to tissue after a period of ischemia or lack of oxygen (anoxia or hypoxia).

The term “infarct growth” as used herein refers to any spreading of tissue damage after the beginning of the ischemic disease. Infarct growth may for example be caused by artery blockages, rupture, mechanical compression, or vasoconstriction. It may be associated with an increase of infarct volume.

Methods for detecting the infarct growth in a subject are well known to the person skilled in the art. For example, magnetic resonance imaging (MRI) or computer tomography (CT) may be used.

Some embodiments also relate to a CD84 inhibitor for use in treating or preventing of ischemic tissue damages, in particular of I/R injury, e.g. during/after organ transplantation. Further embodiments may also relate to treatment or prevention conditions such as postreperfusion syndrome, delayed graft function, loss of graft function or post-transplant lymphoproliferative disorder.

The inventors have been able to show that CD84 inhibitors are capable of inhibiting T cell migration stimulated by CD84 shed from platelets. Consequently, in a preferred embodiment, the CD84 inhibitor according to the first aspect may inhibit T cell migration and/or platelet effects critical for tissue damage in the context of ischemic diseases.

In a further preferred embodiment, the CD84 inhibitor for use of the first aspect as described above may be for preventing the ischemic tissue damage and is administered prior to ischemia.

In an alternative preferred embodiment, the CD84 inhibitor for use of the first aspect as described herein may be used in treating or preventing the ischemic tissue damage, wherein the CD84 inhibitor is administered during or after ischemia.

In this context, the term “preventing” also means preventing a deterioration of the disease.

The route of administration of a CD84 inhibitor may include the typical routes allowing the direct injection in to the blood flow including, for example, orally, intravenously, subcutaneously, intraarterially, by direct injection to the brain, and parenterally. In some embodiments, the CD84 inhibitor for use of the first may be administered orally, intravenously or subcutaneously, in particular intravenously or subcutaneously, in particular preferred intravenously.

In another preferred embodiment, the CD84 inhibitor for use of the first aspect may be used in a method further comprising administering another therapeutic agent.

In general, when the factors are administered in combination, they may be premixed prior to administration, administered simultaneously, or administered singly in series.

The CD84 inhibitor for use of the first aspect may be used in a method further comprising administering one, two, three, four, five or more therapeutic agents.

Suitable other therapeutic agents may be other agents for preventing or treating ischemic tissue damage, ischemic disease, reperfusion injury and/or infarct growth. Further, also therapeutic agents preventing or treating the symptoms or secondary diseases of ischemic tissue damage, ischemic disease, reperfusion injury and/or infarct growth may be administered together with the CD84 inhibitor. Moreover, the CD84 inhibitor for use of the first aspect may be used in a method further comprising administering another therapeutic agent for preventing or treating a disease independent from ischemic tissue damage and/or ischemic disease, reperfusion injury and/or infarct growth.

Suitable other therapeutic agents for preventing or treating ischemic tissue damage and/or ischemic disease, reperfusion injury and/or infarct growth that may be administered together with the CD84 inhibitor according to the first aspect are anticoagulants, immunosuppressants and/or antithrombotics, such as anti-platelet drugs.

For example, antiplatelet-drugs (e.g. aspirin), beta blockers (e.g., carvedilol, propranolol, atenolol), nitroglycerin (for acute relief), immunosuppressants (e.g. FTY720), vasodilators such as calcium channel blockers (e.g., nifedipine (Adalat) and amlodipine), vasosorbide mononitrate, nicorandil and/or recombinant tissue plasminogen activator (rtPA) may be administered together with the CD84 inhibitor according to the first aspect. Further, channel inhibitors (e.g., ivabradine), ACE inhibitors, statins, ranolazine (Ranexa) may also be administered together with the CD84 inhibitor according to the first aspect.

The present invention also relates to an isolated composition for use in treating or preventing of an ischemic disease and/or ischemic tissue damage and/or of an ischemic disease, that comprises a CD84 inhibitor as described above and optionally further therapeutic agents as described above.

The CD84 inhibitor for use may be used in a method further comprising mechanical removal of the blood clot causing the ischemic disease, called mechanical thrombectomy, which is a potential treatment, e.g. for occlusion of a large artery, such as the middle cerebral artery. For example, the CD84 inhibitor for use of the first aspect may be used in a method further comprising thrombolysis, embolectomy, surgical revascularisation, craniectomy and/or partial amputation.

In a second aspect, the invention relates to a use of a CD84 inhibitor for inhibiting CD84 mediated T cell migration in vitro.

The term “T cell migration” as used herein refers to the general movement of T cells, preferably in particular directions to specific locations. Methods for detecting T cell migration are well known to the person skilled in the art. Suitable methods are migration assays, for example based on time-lapse video microscopy.

A preferred embodiment relates to the use of a CD84 inhibitor according to the second aspect, wherein the CD84 inhibitor is as defined in the first aspect.

The invention further relates to a method for treating an ischemic disease and/or ischemic tissue damage, wherein a CD84 inhibitor is administered to a patient in need thereof. All embodiments described above with respect to the first aspect also apply to this aspect.

In a third aspect, the invention relates to a method for screening for a CD84 inhibitor, the method comprising identifying whether a potential CD84 inhibitor inhibits T cell migration.

A “potential CD84 inhibitor” is a molecule that is considered as a candidate for a CD84 inhibitor, but it is not known if partial or complete suppression, inhibition or blocking of the expression, activity or interaction of CD84 occurs. Only after confirming that the potential CD84 inhibitor actually achieves partial or complete suppression, inhibition or blocking of the expression, activity or interaction of CD84, it is considered a CD84 inhibitor. The method in accordance with the third aspect is suitable to identify which candidates achieve this.

In some embodiments of this third aspect, migration of T cells is stimulated with a stimulant. T cells are preferably human T cells or T cells from wildtype (WT) mice, or mice that express the extracellular domain of human CD84 instead of murine CD84. In one embodiment the stimulant may be soluble CD84, in particular recombinant soluble CD84 fused to the Fc part of human IgG1. Preferably, potential CD84 inhibitors that fully or partially suppress the stimulation activity of the stimulant are identified as CD84 inhibitor that inhibit T cell migration.

In some embodiments in accordance with the first aspect, the CD84 inhibitor is selected in accordance with the screening method of the third aspect.

T cell migration may be measured as described above or in the examples, Part 2.

In a preferred embodiment of the third aspect, various CD84 inhibitors may be tested and compared to each other and a CD84 inhibitor is selected which exhibits the highest T cell migration inhibitory effect.

In a fourth aspect, the invention also relates to a method of producing CD84 inhibitors, wherein multiple anti-CD84 antibodies are provided and CD84 inhibitors are selected therefrom in accordance with the above described third aspect.

All embodiments described above with respect to CD84 inhibitor according to the first aspect also apply to the third and fourth aspect.

The invention is not limited to the particular methodology, protocols and reagents described herein because they may vary. Furthermore, the terminology used herein is for the purpose of describing particular embodiments only and is not intended to limit the scope of the present invention. As used herein and in the appended claims, the singular forms “a”, “an”, and “the” include plural reference unless the context clearly dictates otherwise. Similarly, the words “comprise”, “contain” and “encompass” are to be interpreted inclusively rather than exclusively.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, and materials are described herein.

The present invention is further illustrated by the following Figures and Examples, which are intended to explain, but not to limit the invention, and from which further features, embodiments and advantages may be taken. As such, the specific modifications discussed are not to be construed as limitations on the scope of the invention. It will be apparent to the person skilled in the art that various equivalents, changes, and modifications may be made without departing from the scope of the invention, and it is thus to be understood that such equivalent embodiments are to be included herein.

### DESCRIPTION OF THE FIGURES

FIG. 1a: Infarct volumes in mouse brains (cross-section shown) in Cd84^{-/-} mice compared to wild-type (WT) littermates at 24 h after tMCAO as measured by triphenyltetrazolium chloride (TTC) staining. Representative TTC-stained 2 mm brain slices of WT (top row) and Cd84^{-/-} (bottom row) mice at day 1 after tMCAO. The bar on the bottom right represents 1 cm. Chequered regions represent brain regions, which were not stained with TTC, which means that the tissue of this region was affected by the infarct. Non-chequered regions represent brain regions that were stained with TTC and, therefore, were not affected by the infarct.

FIG. 1b: Infarct volumes in mouse brains in Cd84^{-/-} mice compared to wild-type (WT) littermates at 24 h after tMCAO (n=14-15 mice per group). The x-axis represents the two groups of samples (left: WT mice, right: Cd84^{-/-} mice), while the y-axis represents the infarct volume in mm³. Each dot represents a data point, i.e. the data from one mouse. Further marked are the median value as well as the 25^{th} and the 75^{th} percentiles forming bottom and top line of the rectangles. Finally, the lowest and highest value for each sample type is indicated by a line as well. Statistical significances analyzed by Student t test, *p<0.05.

FIG. 1c: Neuroscore of WT and Cd84^{-/-} mice at day 1 after tMCAO (n=14-15 mice per group). Statistical significances analyzed by Student t test, **p<0.01. The x-axis represents the two sample groups: WT mice (left) and Cd84^{-/-} mice (right). The y-axis represents the Neuroscore. Each black dot represents the Neuroscore of a WT mouse, while each circle filled with white color represents the Neuroscore of a Cd84^{-/-} mouse. Medians of each sample group are indicated by a black line. Statistical significances analyzed by Student t test, **p<0.01.

FIG. 2a: Accumulation of lymphocytes and monocytes in the brain after tMCAO. Quantification of brain-infiltrating CD4-positive T lymphocytes in the ipsilateral hemisphere on day 1 after tMCAO of WT (x-axis, left, shaded) and Cd84^{-/-} mice (x-axis, right) (n=5 mice per group). Bar, 100 pm. The y-axis shows the number of ipsilesional CD4⁺ cells. Statistical significances analyzed by Student t test, *p<0.05. Further marked are the median value as well as the 25^{th} and the 75^{th} percentiles forming bottom and top line of the rectangles. Finally, the lowest and highest value for each sample type is indicated by a line as well.

FIG. 2b: Representative quantification of CD11b-positive cells in the ipsilateral hemisphere 24 h after tMCAO of WT (x-axis, left, shaded) and Cd84^{-/-} mice (x-axis, right) (n=5 mice per group). Bar, 100 pm. The y-axis shows the number of ipsilesional CD11b⁺ cells. Further marked are the median value as well as the 25^{th} and the 75^{th} percentiles forming bottom and top line of the rectangles. Finally the lowest and highest value for each sample type is indicated by a line as well.

FIG. 2c: Representative quantification of the percentage of occluded vessels in the infarcted hemispheres of WT (x-axis, left, shaded) and Cd84^{-/-} mice (x-axis, right) (n=5 mice per group). The y-axis shows the number of ipsilesional occluded vessels in percent (%). Statistical significances analyzed by Student t test, ***P<0.001. Further marked are the median value as well as the 25^{th} and the 75^{th} percentiles forming bottom and top line of the rectangles. Finally the lowest and highest value for each sample type is indicated by a line as well.

FIG. 3a: Infarct volumes 24 h after tMCAO of the indicated groups (x-axis) (n=8-12 mice per group). The y-axis represents infarct volume in mm³. Further marked are the median value as well as the 25^{th} and the 75^{th} percentiles forming bottom and top line of the rectangles. Finally, the lowest and highest value for each sample type is indicated by a line as well. Statistical significances analyzed by Student t test, *p<0.05.

FIG. 3b: Neuroscore at day 1 after tMCAO of the indicated groups (x-axis) (n=8-12 mice per group). The y-axis represents the Neuroscore. Further marked are the median value as well as the 25^{th} and the 75^{th} percentiles forming bottom and top line of the rectangles. Finally, the lowest and highest value for each sample type is indicated by a line as well. Statistical significances analyzed by Mann-Whitney U test. *p<0.05.

FIG. 4: Soluble CD84 promotes T cell migration. In the diagrams 4a to 4f below, the x-axis shows the tested groups, while the y-axis represents the distance in pm. Each dot represents the migrated distance over 30 min of one T cell (n=59-80 cells per group of 3-4 independent experiments). For each group the median is indicated by a black line. Statistical significances analyzed by 1-way ANOVA with Bonferroni post hoc test. ***p<0.001. In detail, FIGS. 4a to 4f represent the following:

FIG. 4a: Migrated distance of CD4-positive WT and Cd84^{-/-} T cells treated with vehicle, Phorbol-12-myristate-13-acetate (PMA) or C-C chemokine cysteine motif chemokine ligand 20 (CCL20).

FIG. 4b: WT and Cd84^{-/-} T cell migration in response to stimulation with WT platelet releasate (PLT-R) compared to vehicle.

FIG. 4c: WT T cell migration in response to stimulation with wildtype or Cd84^{-/-} PLT-R compared to control.

FIG. 4d: Migrated distance of CD4-positive WT and Cd84^{-/-} T cells treated with vehicle, recombinant GPVI-Fc or CD84-Fc protein.

FIG. 4e: WT T cell migration in response to stimulation with WT or Cd84^{-/-} PLT-R in the presence of Control-Fc or recombinant CD84-Fc protein.

FIG. 4f: Migrated distance of CD4+ WT and Cd84^{-/-} T cells treated with GPVI-Fc or CD84-Fc on primary murine brain endothelial cells (MBMEC) of WT or Cd84^{-/-} mice.

FIG. 5: Soluble CD84 increases T cell migratory capacity. In the diagrams 5a to 5f below, the x-axis shows the tested groups, while the y-axis represents the speed in pm/min. Each data point represents the speed of migration analyzed over 30 min of one T cell (n=59-80 cells per group of 3-4 independent experiments). For each group the median is indicated by a black line. Statistical significances analyzed by 1-way ANOVA with Bonferroni post hoc test. ***p<0.001. In detail, FIGS. 5a to 5f represent the following:

FIG. 5a: Velocity of WT and Cd84^{-/-} CD4+ T cells treated with vehicle, PMA or C-C chemokine cysteine motif chemokine ligand 20 (CCL20).

FIG. 5b: WT and Cd84^{-/-} T cell migration speed in response to stimulation with WT platelet releasate (PLT-R) compared to vehicle.

FIG. 5c: Speed of WT T cell migration in response to stimulation with WT or Cd84^{-/-} PLT-R compared to vehicle-treated control.

FIG. 5d: Velocity of WT and Cd84^{-/-} CD4+ T cells treated with vehicle, Ctrl-Fc or CD84-Fc protein.

FIG. 5e: WT T cell migration velocity in response to stimulation with WT or Cd84^{-/-} PLT-R in the presence of CD84-Fc or Ctrl-Fc.

FIG. 5f: Velocity of WT (black) and Cd84^{-/-} (white) CD4+ T cells treated with CD84-Fc or Ctrl-Fc on primary murine brain endothelial cells (MBMEC) of WT or Cd84^{-/-} mice.

### EXAMPLES

Part 1—CD84 in Ischemic Disease and Tissue Damage

Materials and Methods

Cohort validation. To assess the transferability of the findings from mouse model into humans, the association of CD84 expression on platelets and stroke severity at day three after hospital admission was analyzed in a subsample of the Stroke-Induced Cardiac FAILure in Mice and Men (SICFAIL) study. SICFAIL is a cohort study, aiming to describe the natural course of cardiac function in 750 unselected acute IS patients (DRKS00011615). The subsample of the SICFAIL study population, analyzed in this study, included 100 patients with the diagnosis of acute IS according to World Health Organisation (WHO) definition (Hatano et al.) aged 18 years or older, who were hospitalized at the stroke unit of university hospital Wuerzburg between June 2016 and January 2017. Written informed consent was obtained prior study enrollment. The Ethics Committee of the Medical Faculty of the University of Wuerzburg (vote176/13) approved the study. All patients or their legal representatives provided written informed consent to participate.

All patients underwent standard treatment and monitoring at stroke unit. Information on stroke severity and acute treatment (intravenous thrombolysis, thrombectomy) was retrieved by review of patient records. Stroke severity was assessed by a neurologist according to National Health Institute Stroke Severity Scale NIHSS (Berger et al.). Additionally, all study participants underwent a personal standardized interview to collect information on demographic characteristics and comorbidities. Poor outcome was defined as NIHSS 5 on day three of hospitalization.

For CD84 analysis, blood was drawn the morning after study enrollment in tubes containing citrate phosphate dextrose adenine. Samples were diluted within 30 min after blood collection in PBS and platelets were stained using CD42b-FITC (SZ2; Immunotech SAS, Marseille, France) and CD84-PE antibodies (CD84.1.21; BioLegend, San Diego, USA). After an incubation period of 30 minutes, the samples were analyzed by flow cytometry instrument FACSCalibur (Becton Dickinson, Franklin Lakes, USA). CD84 expression was defined as mean fluorescence intensity (MFI) of phycoerythrin (PE) in platelets gated by CD42b positivity.

Mice. Animals used in this study were matched for age, sex and genetic background. Experiments were conducted in accordance with the regulations of the local authorities (Regierung von Unterfranken) and performed in accordance with the current ARRIVE (Animal Research: Reporting of In Vivo Experiments) guidelines (https://www.nc3rs.org.uk/arrive-guidelines).

Animal treatment. For T cell transfer experiments into Rag1^{-/-} and Cd84^{-/-} mice, splenic CD4⁺ T cells were isolated by negative selection (Miltenyi Biotech). Cells were injected intravenously (750,000 cells/mouse) 1 day before tMCAO (Kleinschnitz et al. 2010).

Focal ischemia model. Focal cerebral ischemia was induced in 10-to-14-week-old C57BL/6, Cd84^{-/-},²¹ Rag1^{-/-} (Mombaerts et al.), Cd84^{fl/fl,PF4-Cre-neg.} or Cd84^{fl/fl,PF4-Cre} mice by tMCAO as previously described (Schuhmann et al. 2015). Inhalation anesthesia was induced by 2% isoflurane. The duration of the surgical procedure per animal was kept below 10 min. A silicon rubber-coated 6.0 nylon monofilament (6021PK10, Doccol, Redlands, Calif., USA) was advanced through the carotid artery up to the origin of the MCA causing an MCA infarction. After an occlusion time of 60 min, the filament was removed allowing reperfusion. Animals were sacrificed 23 h after reperfusion and brains were checked for intracerebral hemorrhages. Neurologic function was analyzed calculating a Neuroscore (score 0-10) based on the direct sum of the Grip test (score 0-5) and the inverted Bederson score (score 0-5) (Schuhmann et al. 2018, Moran et al., Bederson et al.).

Infarction size measurement. The extent of infarction was quantitatively assessed 24 h after reperfusion. Animals were sacrificed and brains were cut in three 2 mm thick coronal sections. The slices were stained for 20 min at 37° C. with 2% 2,3,5-triphenyltetrazolium chloride (Sigma-Aldrich; 2% weight/volume (w/v) solution) to visualize the infarctions (Junge et al.). Edema-corrected infarct volumes were calculated by planimetry (Image J software, National Institutes of Health) according to the following equation: V_{indirect} (mm³)=V_{infarct}×(1−(VI−VC)/VC). (VI−VC) represents the volume difference between the ischemic hemisphere (VI) and the control hemisphere (VC) and (VI−VC)/VC) expresses this difference as a percentage of the control hemisphere.

Histology. Histology and immunohistochemistry were performed according to standard procedures (Schuhmann et al. 2015). Cryoembedded coronal brain sections (2 mm) were cut into 10-μm-thick slices. Every tenth slice was used for evaluation. The following antibodies were used: monoclonal antibody anti-CD11b (MCA711, Serotec) and polyclonal antibody anti-CD4 (10056, Bio Legend). For quantification of occluded microvessels, brain slices were stained with hematoxylin and eosin. Afterwards the numbers of occluded and opened vessels per hemisphere were counted to determine the percentage of occlusions as previously described. All immunohistologic stainings were analyzed and acquired using a Nikon Eclipse 50i microscope.

Migration assays. The migratory capacity of T cells was measured using ibidi μ-Slides VI0.4 coated with poly-D-lysine (10 μg/mL, Merck) and laminin (20 μg/mL, Merck) or ibidi Glass Bottom μ-Slides 8 well cultured with primary MBMEC as previously described (Bittner et al.). The migration assays were performed in DMEM high glucose (31053-028, Thermo Fischer) with B27 supplement (2%, 17504044, Thermo Fischer) in triplicates for each different condition (PMA (8 μg/μL, Merck), CCL20 (24.5 μg/μL, PeproTech), PLT-R (1:1 diluted with migration media), Fc-proteins (0.2 μg/ml). PLT-R was obtained by stimulating platelets (500000/μL) with 10 μg/mL of the GPVI agonist collagen-related peptide (CRP) for 15 min, subsequently the platelet supernatant was harvested (5 min with 800 g, followed by 5 min with 22000 g, 4° C. The GPVI-Fc fusion protein control-Fc (Gruner et al.), that did not affect T cell migration compared to vehicle (not shown) served as control for CD84-Fc, both Fc fusion proteins were purified in house from transfected HEK cells using standard techniques. For time-lapse video microscopy, MACS isolated CD4⁺ T cells (130-104-454, Miltenyi; 100 cells/μL) were added to the chamber and images were collected every 30 seconds for 30 minutes on a Leica DMI8 inverted microscope with 20×objective. Cells were tracked with LAS X Imaging software and analyzed with ImageJ 1.51 software.

Statistical analyses. All data from animal experiments are given as box plots including median with the 25^{th} percentile, the 75^{th} percentile, minimum and maximum deviation except for the Neuroscore and the migration assays, which are depicted as scatter plots including the mean and standard error of the mean. Human data are presented as median and interquartile range (25^{th} percentile, and 75^{th} percentile) or, in case of categorical data, frequencies and percentages, respectively. Data were tested for Gaussian distribution e.g. with the D'Agostino and Pearson omnibus normality test and then analyzed by Student's t-test, 1-way ANOVA or Mann-Whitney U-test as applicable. In case of frequencies, χ²-test or Fisher's exact test was used to compare groups. Bonferroni correction was applied when comparing more than two groups in the animal experiments. Scores addressing the functional outcome were compared using the Mann-Whitney U-test. Multivariable logistic regression analysis, adjusted for age and NIHSS at baseline, was used to identify the association of CD84 expression and poor outcome on day three of hospitalization (NIHSS 5). P<0.05 was considered statistically significant. For statistical analysis, the GraphPad Prism 5.0 software package (GraphPad Software) and SAS 9.4 (SAS Institute Inc., Cary, N.C., USA) were used.

### Example 1—Involvement of CD84 in Thrombo-Inflammation

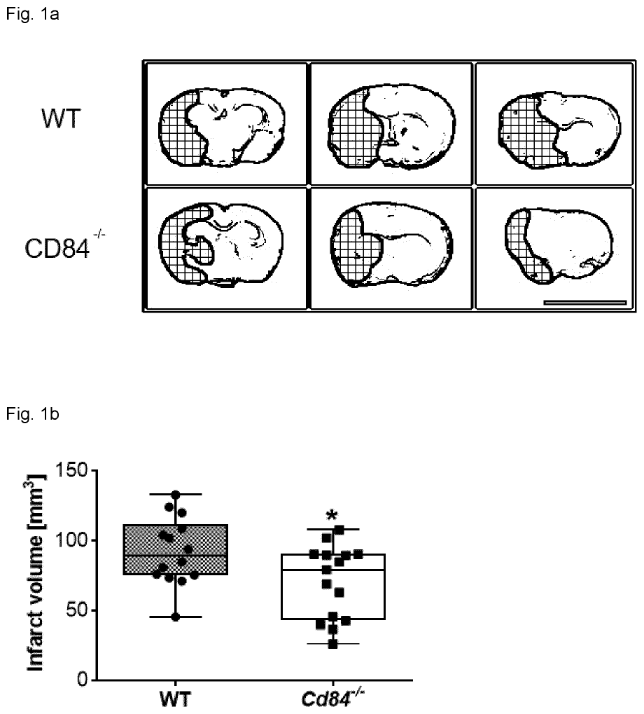

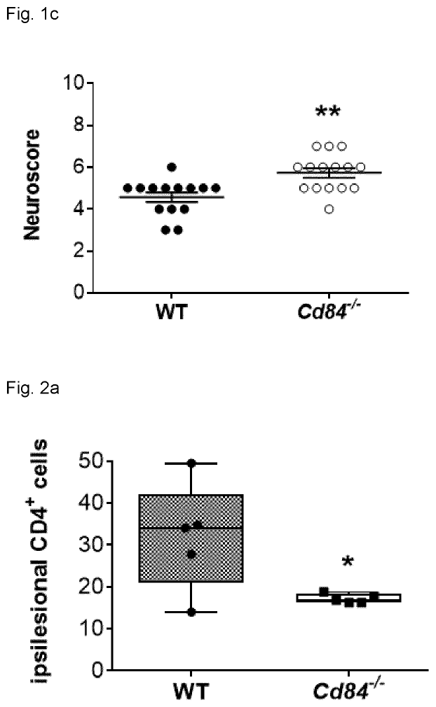

To test for a possible role of CD84 in thrombo-inflammation, Cd84^{-/-} mice were subjected to one hour of transient middle cerebral ischemia (tMCAO) and 23 h of reperfusion. Strikingly, infarct volumes in the mutant mice were significantly reduced compared to wild-type (WT) littermates at 24 h after tMCAO as measured by triphenyltetrazolium chloride (TTC) staining (Med.: 89.3 (25%: 75.1; 75%: 111.6) vs. 79.2 (25%: 42.8; 75%: 90.4) mm³; p<0.05; FIG. 1a, FIG. 1b). The reduction in infarct volumes corresponded with improved neurological outcome in Cd84^{-/-} mice as assessed by the Neuroscore (4.6±0.2 vs. 5.7±0.2, respectively; p<0.01; FIG. 1c).

The results showed that CD84-deficient mice are protected after tMCAO.

### Example 2—Accumulation of Lymphocytes and Monocytes in the Brain After tMCAO

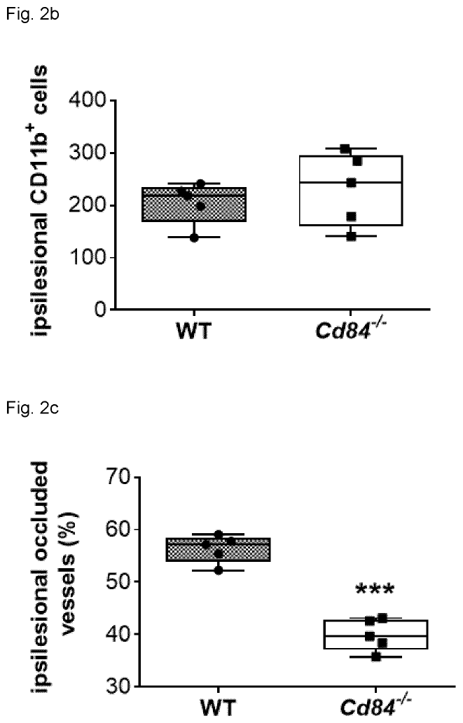

Next, the accumulation of lymphocytes and monocytes in the brain after tMCAO was assessed. Cd84^{-/-} mice showed significantly reduced numbers of CD4⁺ T cells in the infarcted brain (˜50%) compared with WT mice (FIG. 2a), whereas the amount of CD11b⁺ cells (FIG. 2b) was comparable between the two groups. Furthermore, the percentages of occluded vessels in the ipsilateral hemisphere were reduced by ˜35% in Cd84^{-/-} mice 24 h after stroke induction (FIG. 2c). These data demonstrated that CD84-deficiency is protective after tMCAO and that CD84 contributes to T cell recruitment into the infarcted brain territory.

The results indicated that T cell recruitment to the postischemic brain is diminished in CD84-deficient mice.

### Example 3—Relevance of T Cell Expressed CD84 for Infarct Progression

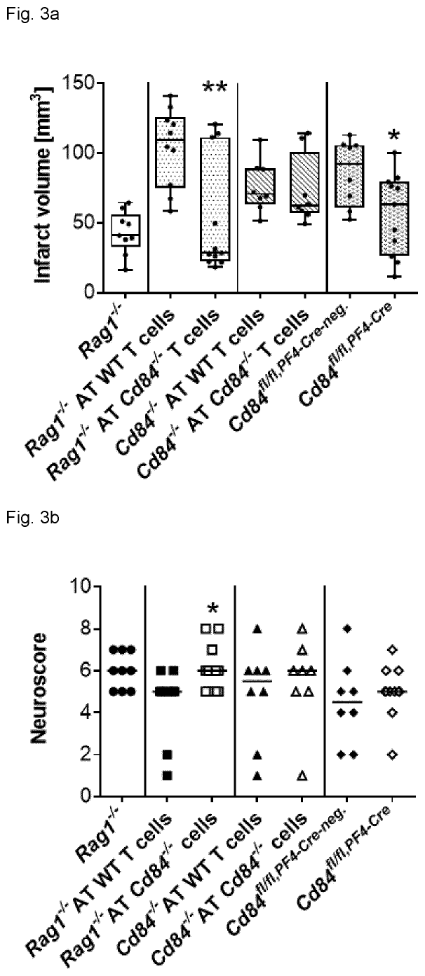

Next, the relevance of T cell expressed CD84 for infarct progression by performing adoptive T cell transfer experiments was assessed. For this, immunodeficient Rag1^{-/-} mice, which lack B and T cells, or Cd84^{-/-} mice received WT or Cd84^{-/-} T cells 24 h before subjecting them to tMCAO. While Rag1^{-/-} displayed small infarcts 24 h after tMCAO (Yilmaz et al., Kleinschnitz et al. 2010, Schuhmann et al. 2017), Rag1^{-/-} mice had fully evolved infarctions after adoptive transfer (AT) of WT T cells (FIG. 3a). In sharp contrast, Rag1^{-/-} mice reconstituted with Cd84^{-/-} T cells had significantly smaller infarcts, as measured by TTC staining (Med.: 100.9 (25%: 74.8; 75%: 126.0) vs. 30.0 (25%: 23.3; 75%: 113.4) mm³; p<0.05; FIG. 3a) and better neurological outcome (FIG. 3b) at day 1 after stroke than those transplanted with WT T cells. However, infarct volumes and functional outcome did not differ when adoptively transferring WT or CD84-deficient T cells into Cd84^{-/-} mice indicating that an interaction of CD84⁺ T cells with another CD84-expressing cell type is required to induce I/R injury.

The results showed that CD84 expression on T cells is required to promote infarct growth after tMCAO.

### Example 4—Role of Platelets

To address a possible role of platelets in this setting, mice with a platelet/megakaryocyte-specific CD84-deficiency (Cd84^{fl/fl,PF4-Cre}; knockout efficacy was confirmed using flow cytometry and Western Blot—not shown) were generated and subjected to tMCAO. These mice developed significantly (˜30%) smaller infarcts at day 1 after tMCAO when compared to littermate control mice (Cd84^{fl/fl}; Med.: 92.3 (25%: 61.0; 75%: 105.9) vs. 63.4 (25%: 26.2; 75%: 79.4) mm³; p<0.05; FIG. 3b) suggesting that platelet CD84 is required for the development of T cell dependent cerebral I/R injury. This is in agreement with the previous observation that platelet depletion abolished detrimental T cell effects during I/R in stroke after adoptive transfer into immune-deficient Rag1^{-/-} mice (Kleinschnitz et al. 2013).

The results showed that CD84 expression on T cells as well as platelet CD84 are required to promote infarct growth after tMCAO.

### Example 5—Requirement of CD84 for T Cell Migration

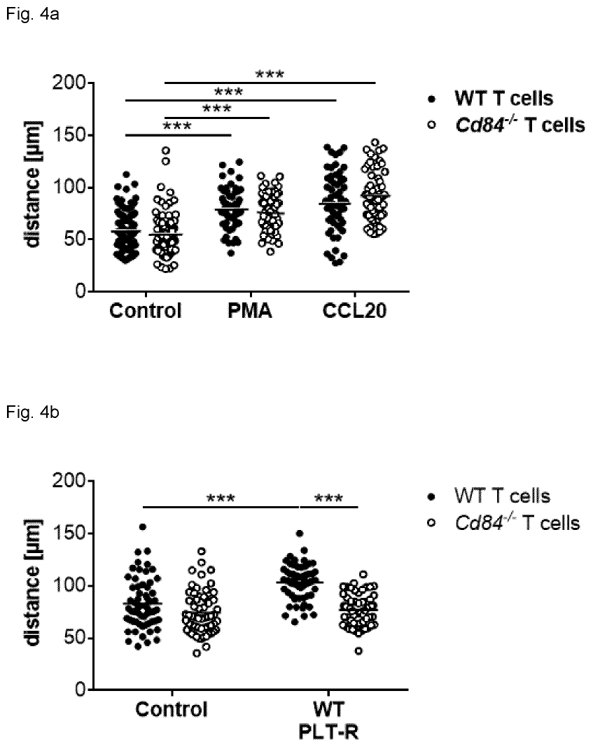

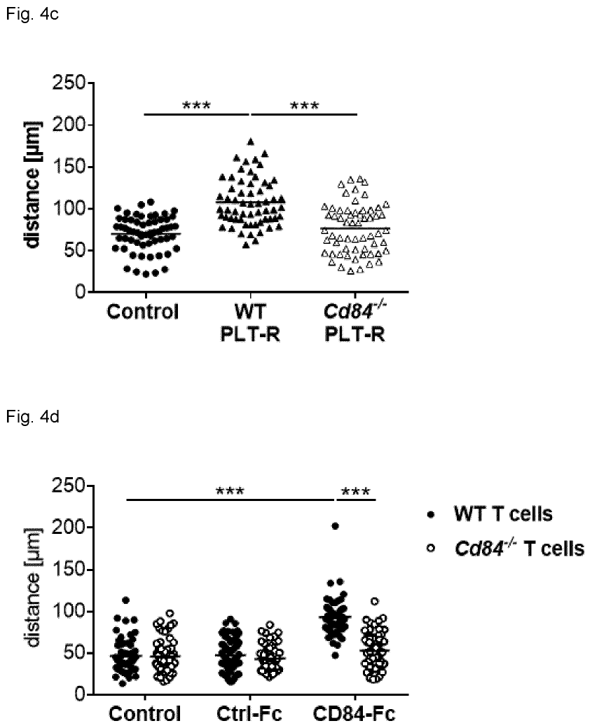

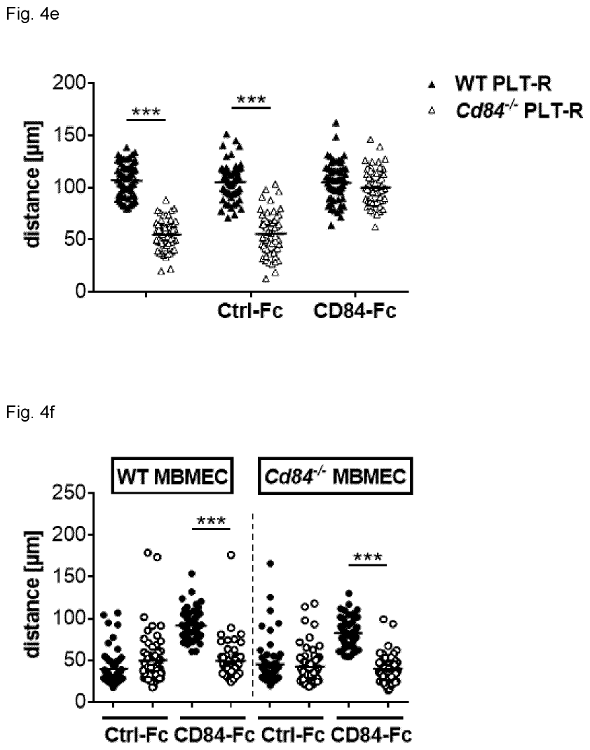

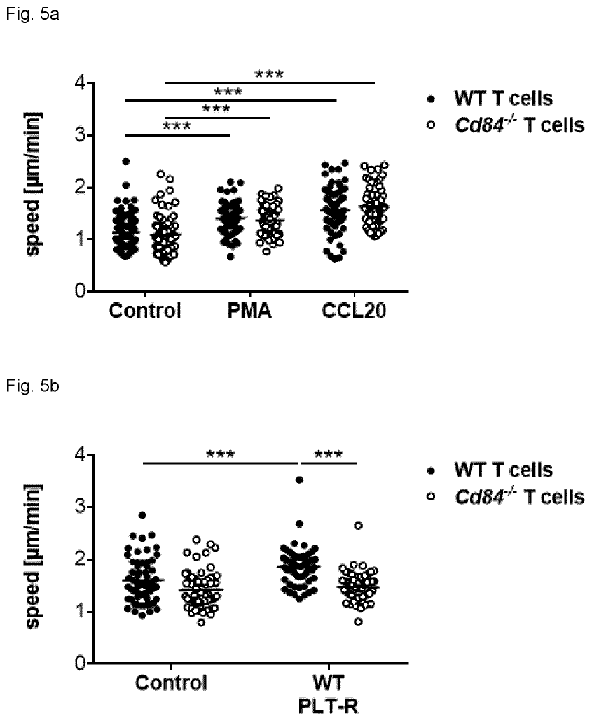

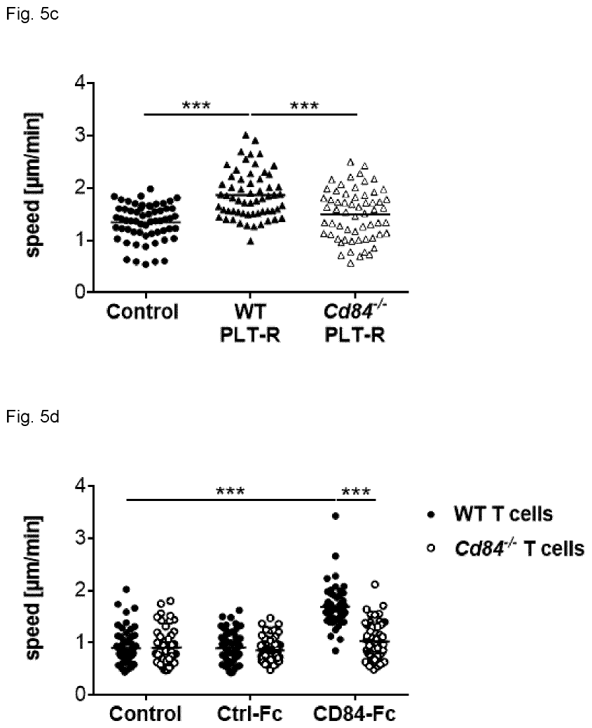

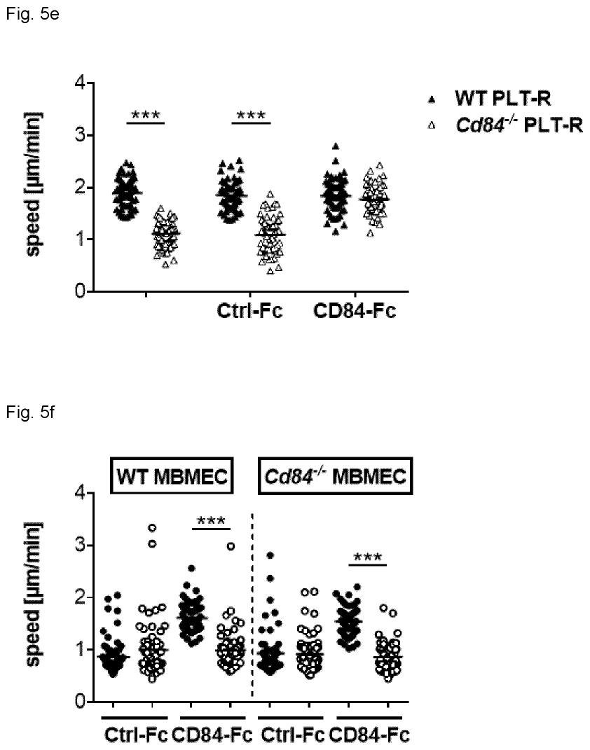

To assess whether CD84 is required for T cell migration, the motility of WT and Cd84^{-/-} T cells in a Laminin/PDL-coated two dimensional in vitro system was measured. Differences between the two genotypes in response to PMA or the C-C chemokine cysteine motif chemokine ligand 20 (CCL20) were not observed (FIG. 4a; 5a). As it was previously shown that the activatory platelet receptor GPVI plays a critical role in cerebral ischemia (Kleinschnitz et al. 2007, Cherpokova et al.), the effect of the releasate from GPVI-stimulated platelets on T cell motility in this assay was tested. Treatment of WT T cells with platelet-releasate (PLT-R) induced an increase ˜25% in the distance and speed of migrating T cells whereas this effect was absent in Cd84^{-/-}T cells (FIG. 4b; 5b).

The releasate of Cd84^{-/-} platelets failed to increase the migratory capacity of WT T cells, suggesting that platelet-derived sCD84 acts as a co-stimulatory factor on T cells (FIG. 4c; 5c). To test this directly, WT and Cd84^{-/-} T cells were treated with recombinant soluble CD84 fused to the Fc part of human IgG1 (CD84-Fc) or a GPVI-Fc protein as control (Ctrl-Fc) and their migratory behavior was compared to that of unstimulated T cells (Control). Notably, WT but not Cd84^{-/-}T cells responded to the presence of CD84-Fc with an increase in velocity and migrated distance compared to the control groups, indicating that the homophilic CD84 interactions are important for the T cell modulating effects of sCD84 (FIG. 4d; 5d). Moreover, Cd84^{-/-} PLT-R supplemented with CD84-Fc restored T cell motility to a level comparable to that induced with WT PLT-R (FIG. 4e; 5e) demonstrating that platelet-derived sCD84 enhances T cell migration. Of note, the migration-promoting effect of sCD84 on WT T cells was also observed in a migration assay in the presence of primary brain derived microvascular endothelial cells, and results were not affected by their genotype (FIG. 4f; 5f).

The results indicated that soluble CD84 promotes T cell migration.

Together, these data demonstrated that platelet CD84 exerts a (co-)stimulatory effect on T cells and is a determinant of infarct progression in a model of ischemic stroke.

### Example 6—CD84 Expression in 100 Participants of the Stroke-Induced Cardiac FAILure in Mice and Men (SICFAIL) Study

Finally, CD84 expression in 100 participants of the Stroke-Induced Cardiac FAILure in Mice and Men (SICFAIL) study was analyzed, to assess a possible link between platelet CD84 expression and disease course in acute ischemic stroke (IS) patients. Median age was 67.0 years (IQR 54.5-76.0 years), 68% of the patients were male and median National Institute of Health Stroke Scale (NIHSS) at admission was 3 (IQR 2-5.5). 19 patients had poor outcome at day three of hospitalization defined as NIHSS≥5. Patients with poor outcome tended to be older, have a history of hypertension and higher NIHSS at admission. Although not statistically significant in univariate analysis (p=0.24), platelet CD84 MFI was identified to be independently associated with poor outcome after adjustment of age and baseline NIHSS (OR=1.27, 95% CI (1.04, 1.57)) (Table 1) in an explanatory multivariable logistic regression approach. These results demonstrate the ability to translate the findings on CD84 in mice into clinical research.

TABLE 1
Multivariable logistic regression to assess the association
of platelet CD84 expression and poor outcome (NIHSS ≥5)
at day three of hospitalization, adjusted for age and NIHSS
at admission.

| | Odds Ratio | (95 % CI) | p-value |
|---|---|---|---|
| Age | 1.07 | (1.01, 1.13) | 0.02 |
| Baseline NIHSS | 1.38 | (1.16, 1.64) | 0.0003 |
| CD84 mean MFI | 1.27 | (1.04, 1.57) | 0.02 |

Part 2—Generation, Selection and Screening of CD84 Activity Blocking Anti-CD84 Antibodies for the Prevention of Cerebral Thrombo-Inflammation

Material and Methods

Mice. Animals used in this study are matched for age, sex and genetic background. Experiments are conducted in accordance with the regulations of the local authorities (Regierung von Unterfranken) and performed in accordance with the current ARRIVE (Animal Research: Reporting of In Vivo Experiments) guidelines (https://www.nc3rs.org.uk/arrive-guidelines).

Focal ischemia model. Focal cerebral ischemia is induced in 10-to-14-week-old C57BL/6 or Cd84^{hCD84/hCD84} mice by tMCAO as previously described (Schuhmann et al. 2015). Inhalation anesthesia is induced by 2% isoflurane. The duration of the surgical procedure per animal is kept below 10 min. A silicon rubber-coated 6.0 nylon monofilament (6021PK10, Doccol, Redlands, Calif., USA) is advanced through the carotid artery up to the origin of the MCA causing an MCA infarction. After an occlusion time of 60 min, the filament is removed allowing reperfusion. Animals are sacrificed 23 h after reperfusion and brains are checked for intracerebral hemorrhages. Neurologic function is analyzed calculating a Neuroscore (score 0-10) based on the direct sum of the Grip test (score 0-5) and the inverted Bederson score (score 0-5) (Schuhmann et al. 2018, Moran et al., Bederson et al.)

Infarction size measurement. The extent of infarction is quantitatively assessed 24 h after reperfusion. Animals were sacrificed and brains are cut in three 2 mm thick coronal sections. The slices are stained for 20 min at 37° C. with 2% 2,3,5-triphenyltetrazolium chloride (Sigma-Aldrich; 2% (w/v) solution) to visualize the infarctions (Junge et al.). Edema-corrected infarct volumes are calculated by planimetry (Image J software, National Institutes of Health) according to the following equation: V_{indirect} (mm³)=V_{infarct}×(1−(VI−VC)/VC). (VI−VC) represents the volume difference between the ischemic hemisphere (VI) and the control hemisphere (VC) and (VI−VC)/VC) expresses this difference as a percentage of the control hemisphere.

Migration assays. The migratory capacity of T cells is measured using ibidi μ-Slides VI0.4 coated with poly-D-lysine (10 μg/mL, Merck) and laminin (20 μg/mL, Merck) or ibidi Glass Bottom μ-Slides 8 well cultured with primary MBMEC as previously described (Bittner et al.). The migration assays are performed in DMEM high glucose (31053-028, Thermo Fischer) with B27 supplement (2%, 17504044, Thermo Fischer) in triplicates for each different condition (anti-CD84 antibodies (10 μg/μL), Fc-proteins (0.2 μg/ml)). A GPVI-Fc fusion protein control-Fc (Gruner et al.), that did not affect T cell migration compared to vehicle (not shown) or a Fc-only protein serves as control for CD84-Fc, all Fc fusion proteins are purified in house from transfected HEK cells using standard techniques. For time-lapse video microscopy, MACS isolated CD4⁺ T cells (130-104-454, Miltenyi; 100 cells/μL) are added to the chamber and images are collected every 30 seconds for 30 minutes on a Leica DM18 inverted microscope with 20×objective. Cells are tracked with LAS X Imaging software and analyzed with ImageJ 1.51 software.

Statistical analyses. Data are tested for Gaussian distribution e.g. with the D'Agostino and Pearson omnibus normality test and then analyzed by Student's t-test, 1-way ANOVA or Mann-Whitney U-test as applicable. Bonferroni correction is applied when comparing more than two groups in the animal experiments. Scores addressing the functional outcome are compared using the Mann-Whitney U-test. P<0.05 is considered statistically significant. For statistical analysis, the GraphPad Prism 5.0 software package (GraphPad Software) and SAS 9.4 (SAS Institute Inc., Cary, N.C., USA) are used.

### Example 7—Generation of Anti-CD84 Antibodies

Cd84^{-/-} mice, 6-8 weeks of age, are immunized with a CD84 immuno-precipitate from mouse or human platelet lysate. Each antigen is resolved in Freund's adjuvant and injected subcutaneously. The immunizations are performed repeatedly, before splenic B cells of an immunized mouse are harvested and fused with myeloma cells. The successfully fused Hybridoma cells are positively selected using a specific selection medium. The supernatants of antibody-producing hybridoma cells are screened for the presence of antibodies that recognize murine or human CD84 by enzyme-linked immunosorbent assays (ELISA) and/or flow cytometry.

### Example 8—Studying the Effect of Anti-CD84 Antibodies on T Cell Motility; Selection of Blocking Anti-CD84 Antibodies as CD84 Inhibitors

To assess whether anti-CD84 antibodies affect the motility enhancing effect of soluble CD84 (sCD84) the motility of T cells is assessed in a Laminin/PDL-coated two dimensional in vitro system. Human T cells or T cells from wildtype (WT), or Cd84^{hCD84/hCD84} mice (mice that express the extracellular domain of human CD84 instead of murine CD84) are treated with recombinant soluble CD84 fused to the Fc part of human IgG1 (CD84-Fc) or control-Fc protein with or without anti-CD84 antibodies. The migratory behavior of these T cells is analyzed and compared with that of unstimulated T cells (control).

CD84-Fc should trigger an increase in velocity and migrated distance compared to the control groups, and this mobility promoting effect of CD84-Fc should be reduced by blocking anti-CD84 antibodies. Non-blocking anti-CD84 antibodies that do not reduce mobility-promoting effect of CD84-Fc are discarded and blocking anti-CD84 antibodies that significantly reduce mobility promoting effect of CD84-Fc are selected as inhibitors of CD84 activity for further experiments.

### Example 9—Studying the Effect of Blocking Anti-CD84 Antibodies on Experimental Stroke

To confirm the effect of the anti-CD84 antibodies in thrombo-inflammation, wild-type (WT) or Cd84^{hCD84/hCD84} mice (mice that express the extracellular domain of human CD84 instead of murine CD84) receive anti-CD84 IgG or Fab or F(ab′)₂ fragments thereof (10-100 μg/mouse) before, during or after being subjected to one hour of transient middle cerebral ischemia (tMCAO) and 23 h of reperfusion. 24 h after tMCAO, the neurological outcome in anti-CD84 treated mice will be compared with that of control animals (mice of the same genotype that received control IgG (Fab or F(ab′)₂ fragments thereof) or vehicle instead of anti-CD84 antibodies (Fab or F(ab′)₂ fragments) by assessing the Neuroscore. In addition, infarct volumes of the mice are measured by triphenyltetrazolium chloride (TTC) staining. The expectation is that anti-CD84 antibodies or Fab or F(ab′)₂ fragments thereof that block the interaction between soluble CD84 and CD84 on T cells result in smaller infarct sizes and a better neurological outcome.

### REFERENCES

Berkhemer, O. A., et al. A randomized trial of intraarterial treatment for acute ischemic stroke. N. Engl. J. Med. 372, 11-20 (2015).

Global Burden of Disease Study, C. Global, regional, and national incidence, prevalence, and years lived with disability for 301 acute and chronic diseases and injuries in 188 countries, 1990-2013: a systematic analysis for the Global Burden of Disease Study 2013. Lancet 386, 743-800 (2015).

Mizuma, A., You, J. S. & Yenari, M. A. Targeting Reperfusion Injury in the Age of Mechanical Thrombectomy. Stroke 49, 1796-1802 (2018).

Goyal, M., et al. Endovascular thrombectomy after large-vessel ischaemic stroke: a meta-analysis of individual patient data from five randomised trials. Lancet 387, 1723-1731 (2016).

Church, E. W., Gundersen, A., Glantz, M. J. & Simon, S. D. Number needed to treat for stroke thrombectomy based on a systematic review and meta-analysis. Clin. Neurol. Neurosurg. 156, 83-88 (2017).

Hallenbeck, J. M. & Dutka, A. J. Background review and current concepts of reperfusion injury. Arch. Neurol. 47, 1245-1254 (1990).

Eltzschig, H. K. & Eckle, T. Ischemia and reperfusion--from mechanism to translation. Nature medicine 17, 1391-1401 (2011).

Yilmaz, G., Arumugam, T. V., Stokes, K. Y. & Granger, D. N. Role of T lymphocytes and interferon-gamma in ischemic stroke. Circulation 113, 2105-2112 (2006).

Kleinschnitz, C., et al. Early detrimental T-cell effects in experimental cerebral ischemia are neither related to adaptive immunity nor thrombus formation. Blood 115, 3835-3842 (2010).

Kleinschnitz, C., et al. Targeting platelets in acute experimental stroke: impact of glycoprotein Ib, VI, and IIb/IIIa blockade on infarct size, functional outcome, and intracranial bleeding. Circulation 115, 2323-2330 (2007).

Kleinschnitz, C., et al. Regulatory T cells are strong promoters of acute ischemic stroke in mice by inducing dysfunction of the cerebral microvasculature. Blood 121, 679-691 (2013).

Stoll, G., Kleinschnitz, C. & Nieswandt, B. Combating innate inflammation: a new paradigm for acute treatment of stroke? Annals of the New York Academy of Sciences 1207, 149-154 (2010).

Stoll, G. & Nieswandt, B. Thrombo-inflammation in acute ischaemic stroke—implications for treatment. Nature Reviews Neurology In press(2019).

Nieswandt, B., Kleinschnitz, C. & Stoll, G. Ischaemic stroke: a thrombo-inflammatory disease? The Journal of physiology 589, 4115-4123 (2011).

Nanda, N. & Phillips, D. R. Novel targets for antithrombotic drug discovery. Blood cells, molecules & diseases 36, 228-231 (2006).

Martin, M., et al. CD84 functions as a homophilic adhesion molecule and enhances IFN-gamma secretion: adhesion is mediated by Ig-like domain 1. J. Immunol. 167, 3668-3676 (2001).

Romero, X., et al. Differential expression of SAP and EAT-2-binding leukocyte cell-surface molecules CD84, CD150 (SLAM), CD229 (Ly9) and CD244 (2B4). Tissue antigens 64, 132-144 (2004).

Nanda, N., et al. Platelet aggregation induces platelet aggregate stability via SLAM family receptor signaling. Blood 106, 3028-3034 (2005).

Tangye, S. G., Nichols, K. E., Hare, N. J. & van de Weerdt, B. C. Functional requirements for interactions between CD84 and Src homology 2 domain-containing proteins and their contribution to human T cell activation. J Immunol 171, 2485-2495 (2003).

Cannons, J. L., et al. Optimal germinal center responses require a multistage T cell:B cell adhesion process involving integrins, SLAM-associated protein, and CD84. Immunity. 32, 253-265 (2010).

Hofmann, S. Studies on the function and regulation of CD84, GPVI and Orai2 in genetically modified mice. Doctoral thesis, Wurzburg (2013).

Hofmann, S., Vogtle, T., Bender, M., Rose-John, S. & Nieswandt, B. The SLAM family member CD84 is regulated by ADAM10 and calpain in platelets. Journal of thrombosis and haemostasis: JTH 10, 2581-2592 (2012).

Hofmann, S., et al. Mice Lacking the SLAM Family Member CD84 Display Unaltered Platelet Function in Hemostasis and Thrombosis. PLoS ONE 9, e115306 (2014).

Tatusova et al. BLAST 2 Sequences, a new tool for comparing protein and nucleotide sequences. FEMS Microbiol. Lett. 174: 247-250 (1999).

Segal D M et al. Bispecific antibodies in cancer therapy., Current Opin. Immunol., 11:558-562 (1999).

Van Spriel A B et al. Immunotherapeutic perspective for bispecific antibodies, Immunology Today, 21:391-397 (2000).

P. J. Lachmann, S. Songsivilai, Bispecific antibody: a tool for diagnosis and treatment of disease., Clin. Exp. Immunol., 79: 315 (1990).

V. Raso, T. Griffin, Hybrid Antibodies with Dual Specificity for the Delivery of Ricin to Immunoglobulin-bearing Target, CellsCancer Res., 41:2073 (1981).

S. Honda et al., A hyman hybrid hybridoma producing a bispecific monoclonal antibody that can target tumor cells for attack by Pseudomonas aeruginosa exotoxin A, Cytotechnology, 4:59 (1990).

J. Corvalan et al., Tumour therapy with vinca alkaloids targeted by a hybrid-hybrid monoclonal antibody recognising both CEA and vinca alkaloids, Intl. J. Cancer Suppl., 2:22 (1988).

M. Pimm et al., A bispecific monoclonal antibody against methotrexate and a human tumour associated antigen augments cytotoxicity of methotrexate-carrier conjugate, British J. of Cancer, 61:508 (1990).

M. Brennan et al., Preparation of bispecific antibodies by chemical recombination of monoclonal immunoglobulin G1 fragments., Science 229:81 (1985).

C. Milstein and A. C. Cuello, Hybrid hybridomas and their use in immunohistochemistry, Nature, 305: 537-539 (1983).

Bird R E, et al., Single-chain antigen-binding proteins, Science 242:423-426 (1988).

Orlandi et al., Cloning immunoglobulin variable domains for expression by the polymerase chain reaction. Proc Natl Acad Sci USA 86:3833-3837 (1989).

Clarkson et al., Making antibody fragments using phage display libraries., Nature 352: 624-628 (1991).

Hatano, S. Experience from a multicentre stroke register: a preliminary report. Bull. World Health Organ. 54, 541-553 (1976).

Berger, K., et al. [The reliability of stroke scales. The german version of NIHSS, ESS and Rankin scales]. Fortschr. Neurol. Psychiatr. 67, 81-93 (1999).

Mombaerts, P., et al. RAG-1-deficient mice have no mature B and T lymphocytes. Cell 68, 869-877 (1992).

Schuhmann, M. K., et al. CD28 superagonist-mediated boost of regulatory T cells increases thrombo-inflammation and ischemic neurodegeneration during the acute phase of experimental stroke. J. Cereb. Blood Flow Metab. 35, 6-10 (2015).

Schuhmann, M. K., et al. Influence of Thrombolysis on the Safety and Efficacy of Blocking Platelet Adhesion or Secretory Activity in Acute Ischemic Stroke in Mice. Translational stroke research 9, 493-498 (2018).

Moran, P. M., Higgins, L. S., Cordell, B. & Moser, P. C. Age-related learning deficits in transgenic mice expressing the 751-amino acid isoform of human beta-amyloid precursor protein. Proc. Natl. Acad. Sci. U.S.A. 92, 5341-5345 (1995).

Bederson, J. B., et al. Rat middle cerebral artery occlusion: evaluation of the model and development of a neurologic examination. Stroke 17, 472-476 (1986).

Junge, C. E., et al. The contribution of protease-activated receptor 1 to neuronal damage caused by transient focal cerebral ischemia. Proceedings of the National Academy of Sciences of the United States of America 100, 13019-13024 (2003).

Bittner, S., et al. Endothelial TWIK-related potassium channel-1 (TREK1) regulates immune-cell trafficking into the CNS. Nat. Med. 19, 1161-1165 (2013).

Gruner, S., et al. Relative antithrombotic effect of soluble GPVI dimer compared with anti-GPVI antibodies in mice. Blood 105, 1492-1499 (2005).

Schuhmann, M. K., Langhauser, F., Kraft, P. & Kleinschnitz, C. B cells do not have a major pathophysiologic role in acute ischemic stroke in mice. J. Neuroinflammation 14, 112 (2017).

Cherpokova, D., et al. SLAP/SLAP2 prevent excessive platelet (hem)ITAM-signaling in thrombosis and ischemic stroke in mice. Blood 125, 185-194 (2015).

Schuhmann, M. K., et al. Influence of Thrombolysis on the Safety and Efficacy of Blocking Platelet Adhesion or Secretory Activity in Acute Ischemic Stroke in Mice. Translational stroke research 9, 493-498 (2018).

Moran, P. M., Higgins, L. S., Cordell, B. & Moser, P. C. Age-related learning deficits in transgenic mice expressing the 751-amino acid isoform of human beta-amyloid precursor protein. Proc. Natl. Acad. Sci. U.S.A. 92, 5341-5345 (1995).

1. A CD84 inhibitor for use in treating or preventing of an ischemic disease and/or ischemic tissue damage.

2. The CD84 inhibitor for use according to claim 1, wherein the inhibitor is selected from the group consisting of an antibody, a blocking polypeptide, a small molecule, or an inhibitory nucleic acid, in particular wherein the inhibitor is an antibody.

3. The CD84 inhibitor for use according to claim 2, wherein the antibody is selected from the group consisting of a monoclonal antibody, a chimeric antibody, a humanized antibody, a human antibody, a bispecific antibody, an scFv, a multimer of an scFv, a tandab, a diabody, triabody, a flexibody, or a fragment thereof.

4. The CD84 inhibitor for use according to any of claims 1 to 3, wherein the CD84 inhibitor is capable of inhibiting homotypic receptor interaction and/or dimerization of CD84 molecules.

5. The CD84 inhibitor for use according to any of claims 1 to 4, wherein the CD84 inhibitor inhibits the interaction of a CD84 molecule on T cells with another CD84 molecule derived from platelets.

6. The CD84 inhibitor for use according to any of claims 1 to 5, wherein the CD84 inhibitor binds to the domain of CD84 that mediates homotypic receptor interaction of CD84.

7. The CD84 inhibitor for use according to any of claims 1 to 6, wherein the ischemic disease and/or ischemic tissue damage is selected from the group consisting of trauma, ischemic cerebrovascular disorder, in particular cerebral infarction, ischemic renal disease, ischemic pulmonary disease, infection-related ischemic disease, ischemic disease of limbs, ischemic heart disease, myocardial infarction, atherosclerosis, peripheral vascular disorder, a pulmonary embolus, a venous thrombosis, a transient ischemic attack, unstable angina, cerebral vascular ischemia, stroke, an ischemic neurological disorder, ischemic kidney disease, vasculitis, transplantation, endarterectomy, aneurysm repair surgery, an inflammatory disorder, hypothermia or traumatic injury.

8. The CD84 inhibitor for use according to any of claims 1 to 7, wherein the method is for treating or preventing ischemia/reperfusion injury and/or infarct growth.

9. The CD84 inhibitor for use according to any of claims 1 to 8, wherein the CD84 inhibitor is for preventing the ischemic disease and wherein the CD84 inhibitor is administered prior to ischemia.

10. The CD84 inhibitor for use according to any of claims 1 to 8, wherein the method is for treating or preventing the ischemic disease, and wherein the CD84 inhibitor is administered during or after ischemia.

11. The CD84 inhibitor for use according to any of claims 1 to 10, wherein the CD84 inhibitor is capable of inhibiting CD84 mediated T cell migration.

12. The CD84 inhibitor for use according to any of claims 1 to 11, wherein the method further comprises administering another therapeutic agent.

13. The CD84 inhibitor for use according to any of claims 1 to 12, wherein the method further comprises suppression of T cell migration with said CD84 inhibitor, in particular wherein the ischemic disease and/or ischemic tissue damage is at least partially mediated by T cell migration.

14. Use of a CD84 inhibitor for inhibiting CD84 mediated T cell migration in vitro.

15. The use according to claim 14, wherein the CD84 inhibitor is as defined in any of claims 2 to 13.

16. A method for screening for a CD84 inhibitor, in particular for a CD84 inhibitor as defined in any of claims 2 to 13, the method comprising identifying whether a potential CD84 inhibitor inhibits T cell migration.


SEQUENCE LISTING (4 sequences)

SEQ ID NO: 1 - protein, 345 aa, Homo sapiens
MAQHHLWILLLCLQTWPEAAGKDSEIFTVNGILGESVTFPVNIQEPRQVKIIAWTSKTSVAYVTPGDSETAPVVTVTHRNYYERIHALGPNYNLVISDLRMEDAGDYKADINTQADPYTTTKRYNLQIYRRLGKPKITQSLMASVNSTCNVTLTCSVEKEEKNVTYNWSPLGEEGNVLQIFQTPEDQELTYTCTAQNPVSNNSDSISARQLCADIAMGFRTHHTGLLSVLAMFFLLVLILSSVFLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 2 - protein, 329 aa, Mus musculus
MAQRHLWIWFLCLQTWSEAAGKDADPVVMNGILGESVTFLLNIQEPKKIDNIAWTSQSSVAFIKPGVNKAEVTITQGTYKGRIEIIDQKYDLVIRDLRMEDAGTYKADINEENEETITKIYYLHIYRRLKTPKITQSLISSLNNTCNITLTCSVEKEEKDVTYSWSPFGEKSNVLQIVHSPMDQKLTYTCTAQNPVSNSSDSVTVQQPCTDTPSFHPRHAVLPGGLAVLFLLILIPMLAFLFRLYKRRRDRIVLEADDVSKKTVYAVVSRNAQPTESRIYDEIPQSKMLSCKKDPVTTIYSSVQLSEKMKETNMKDRSLPKALGNEIVV

SEQ ID NO: 3 - protein, 99 aa, Homo sapiens
GILGESVTFPVNIQEPRQVKIIAWTSKTSVAYVTPGDSETAPVVTVTHRNYYERIHALGPNYNLVISDLRMEDAGDYKADINTQADPYTTTKRYNLQIY

SEQ ID NO: 4 - protein, 96 aa, Mus musculus
GILGESVTFLLNIQEPKKIDNIAWTSQSSVAFIKPGVNKAEVTITQGTYKGRIEIIDQKYDLVIRDLRMEDAGTYKADINEENEETITKIYYLHIY